(19) United States
(12) Patent Application Publication
(10) Pub. No.: US 2024/0149056 A1
(43) Pub. Date: May 9, 2024

(54) ELECTRODE PHYSICAL MANAGEMENT TECHNOLOGIES

(71) Applicant: COCHLEAR LIMITED, Macquarie University, NSW (AU)

(72) Inventors: Paul Michael CARTER, Macquarie University (AU); Nicholas Charles Kendall PAWSEY, Macquarie University (AU); Christopher Joseph LONG, Macquarie University (AU); Sara Ingrid DURAN, Macquarie University (AU); Peter GIBSON, Macquarie University (AU)

(21) Appl. No.: 18/282,251

(22) PCT Filed: Mar. 25, 2021
(86) PCT No.: PCT/IB2022/052767

§ 371 Date: Sep. 15, 2023

Related U.S. Application Data
(60) Provisional application No. 63/165,939, filed on Mar. 25, 2021.

(51) Int. Cl. A61N 1/05; A61N 1/36; G16H 10/40; G16H 40/63

(52) U.S. Cl. CPC A61N 1/0541; A61N 1/36038; G16H 10/40; G16H 40/63

(57) ABSTRACT

A medical device, including an implantable portion of the medical device, the implantable portion including at least one electrode, wherein the implantable portion is configured to, while implanted in a human, obtain data indicative of wear of the at least one electrode. In an exemplary embodiment, the medical device is a cochlear implant.

### CROSS-REFERENCE TO RELATED APPLICATIONS

This application claims priority to U.S. Provisional Application No. 63/165,939, entitled ELECTRODE PHYSICAL MANAGEMENT TECHNOLOGIES, filed on Mar. 25, 2021, naming Paul Michael CARTER of Macquarie University, Australia as an inventor, the entire contents of that application being incorporated herein by reference in its entirety.

### BACKGROUND

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In an exemplary embodiment, there is a medical device, comprising an implantable portion of the medical device, the implantable portion including at least one electrode, wherein the implantable portion is configured to, while implanted in a human, obtain data indicative of wear of the at least one electrode.

In an exemplary embodiment, there is an implantable electrode array, comprising a plurality of electrodes and a carrier carrying the plurality of electrodes, wherein the implantable electrode array is configured to enable in vivo analysis of a wear status of at least one electrode of the plurality of electrodes.

In an exemplary embodiment, there is a method comprising obtaining data relating to a phenomenon internal to a human having an electrode array implanted in the human and analyzing the obtained data to determine a wear status and/or a passive dissolution and/or wear rate of at least one electrode of the electrode array, wherein the action of obtaining data is executed, at the time of obtaining data, non-invasively and/or minimally invasively.

In an exemplary embodiment, there is a method, comprising obtaining data relating to a current and/or future wear rate and/or current and/or future wear status of an implanted electrode implanted in a human, the implanted electrode being part of a medical device prosthesis used by the human, analyzing the obtained data, and based on results of the analyzing, least one of: (i) identifying an adjustment of an operational parameter of the medical device prosthesis to change a future rate of wear; (ii) prescribing a substance to be taken by the human to slow the future rate of wear and/or proscribing a substance to be taken by the human that has an effect on the future rate of wear; (iii) instructing the human to use the medical device prosthesis in a different manner; or (iv) taking no action.

In an exemplary embodiment, there is a method, comprising obtaining access to data related to a human who has a medical device implant including electrodes implanted therein and based on the obtained data, assessing a risk level of deleterious passive dissolution and/or active dissolution of an implanted electrode implanted in a human based on at least one of: a size and/or shape of the cochlea;
a lifestyle of the human;
composition of perilymph;
diseases and/or co-morbidities and/or associated treatments;
cochlear implant design and/or cochlear implant surgical factors;
electrode type; or
electrode position in the cochlea.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIGS. 1B-1D are quasi functional diagrams of an exemplary device to which some embodiments may be applicable;

FIGS. 1E and 2A and 2B and 2C and 1F present some schematics related to base technologies associated with some embodiments;

FIGS. 3-8 and 10-12 and 15 and 16 depict various schematics of various embodiments of electrodes according to some embodiments;

FIGS. 9 and 13 and 14A and 14B and 15 present various charts of electrical phenomenon that are applicable to some embodiments;

FIGS. 17 and 18 and 20 and 21 and 22 present exemplary flowcharts for exemplary methods according to some exemplary embodiments; and

FIG. 19 presents an exemplary diagram of some electrode arrays with some conceptual concepts presented therewith.

### DETAILED DESCRIPTION

Merely for ease of description, the techniques presented herein are primarily described herein with reference to an illustrative medical device, namely a hearing prosthesis. First introduced is a cochlear implant. The techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, electroporation, etc., and thus any disclosure herein is a disclosure of utilizing such devices with the teachings herein, providing that the art enables such. The teachings herein can also be used with conventional hearing devices, such as telephones and ear bud devices connected MP3 players or smart phones or other types of devices that can provide audio signal output. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

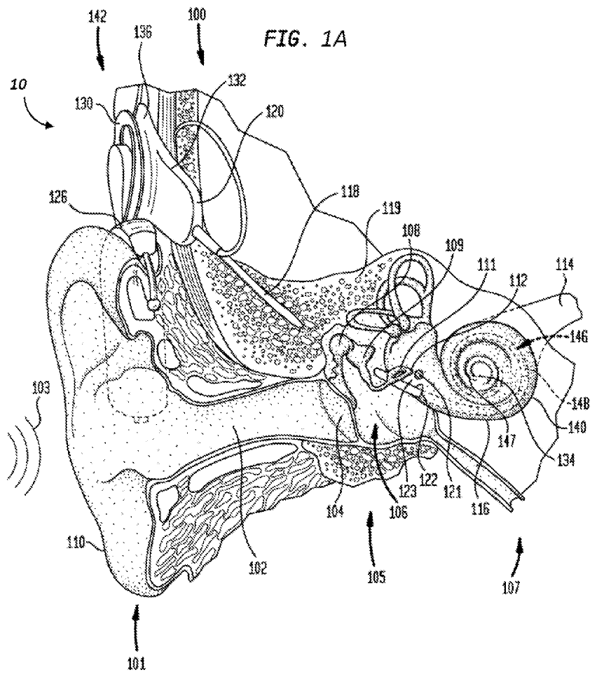

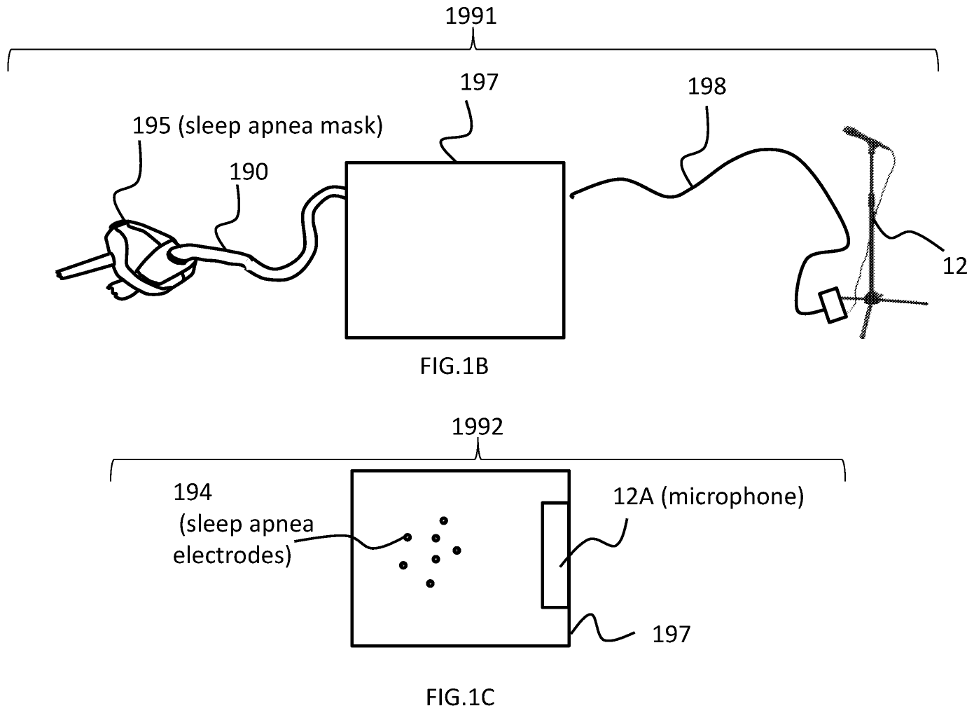

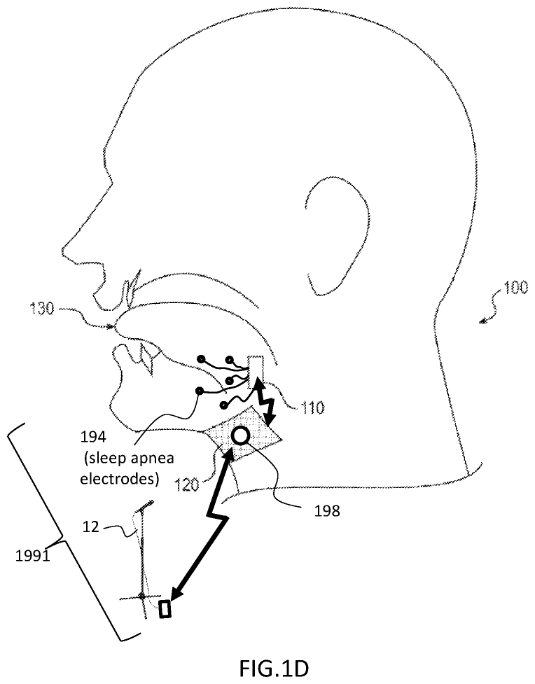

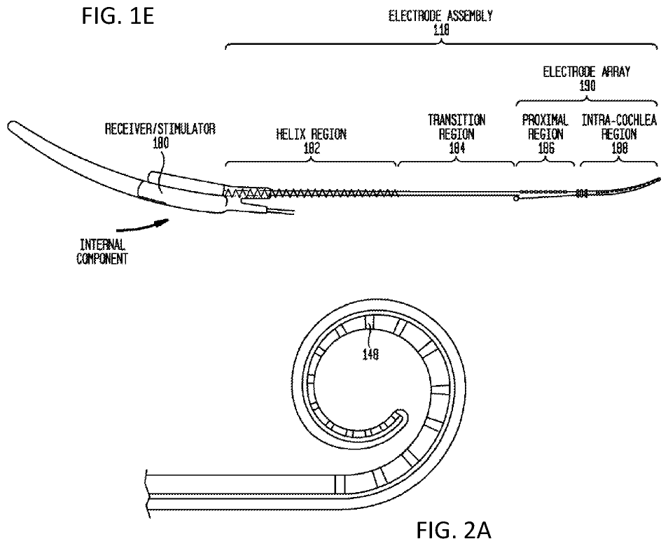

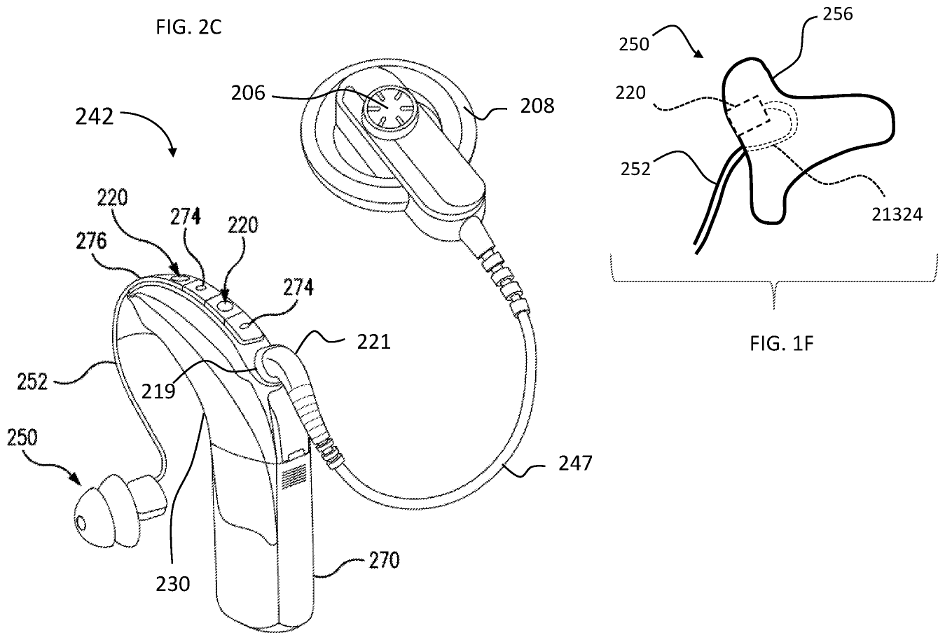

FIG. 1A is perspective view of a partially implantable cochlear implant, referred to as cochlear implant 100, implanted in a recipient. The cochlear implant 100 is part of a system 10 that can include external component(s), as will be detailed below.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1A with an external device 142, that is part of system 10 (along with cochlear implant 100), which, as described below, is configured to provide power to the cochlear implant.

In the illustrative arrangement of FIG. 1A, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive, and inductive transfer, may be used to transfer the power and/or data from external device 142 to cochlear implant 100. In the illustrative embodiments of FIG. 1A, the external energy transfer assembly comprises an external coil 130 that forms part of an inductive radio communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1A is merely illustrative, and other external devices may be used with embodiments of the present invention.

Cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate stimulating assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate stimulating assembly 118.

Elongate stimulating assembly 118 has a proximal end connected to main implantable component 120, and a distal end implanted in cochlea 140. Stimulating assembly 118 extends from main implantable component 120 to cochlea 140 through mastoid bone 119. In some embodiments stimulating assembly 118 may be implanted at least in basal region 116, and sometimes further. For example, stimulating assembly 118 may extend towards apical end of cochlea 140, referred to as cochlea apex 134. In certain circumstances, stimulating assembly 118 may be inserted into cochlea 140 via a cochleostomy 122. In other circumstances, a cochleostomy may be formed through round window 121, oval window 112, the promontory 123 or through an apical turn 147 of cochlea 140.

Stimulating assembly 118 comprises a longitudinally aligned and distally extending array 146 of electrodes 148, disposed along a length thereof. As noted, a stimulator unit generates stimulation signals which are applied by stimulating contacts 148, which, in an exemplary embodiment, are electrodes, to cochlea 140, thereby stimulating auditory nerve 114. In an exemplary embodiment, stimulation contacts can be any type of component that stimulates the cochlea (e.g., mechanical components, such as piezoelectric devices that move or vibrate, thus stimulating the cochlea (e.g., by inducing movement of the fluid in the cochlea), electrodes that apply current to the cochlea, etc.). Embodiments detailed herein will generally be described in terms of an electrode assembly 118 utilizing electrodes as elements 148. It is noted that alternate embodiments can utilize other types of stimulating devices. Any device, system, or method of stimulating the cochlea via a device that is located in the cochlea can be utilized in at least some embodiments. In this regard, any implantable array that stimulates tissue, such as a retinal implant array, or a spinal array, or a pacemaker array, etc., is encompassed within the teachings herein unless otherwise noted.

As noted, cochlear implant 100 comprises a partially implantable prosthesis, as contrasted to a totally implantable prosthesis that is capable of operating, at least for a period of time, without the need for external device 142. Therefore, cochlear implant 100 does not comprise a rechargeable power source that stores power received from external device 142, as contrasted to an embodiment where there is an implantable rechargeable power source (e.g., a rechargeable battery). During operation of cochlear implant 100, the power is transferred from the external component to the implanted component via the link, and distributed to the various other implanted components as needed.

It is noted that the teachings detailed herein and/or variations thereof can be utilized with a totally implantable prosthesis. That is, in an alternate embodiment of the cochlear implants or other hearing prostheses detailed herein, the prostheses are totally implantable prostheses, such as where there is an implanted microphone and sound processor and battery.

FIG. 1B provides a schematic of an exemplary conceptual sleep apnea system 1991. Here, this exemplary sleep apnea system utilizes a microphone 12 (represented conceptually) to capture a person's breathing or otherwise the sounds made by a person while sleeping. The microphone transduces the captured sound into an electrical signal which is provided via electrical leads 198 to the main unit 197, which includes a processor unit that can evaluate the signal from leads 198 or, in another arrangement, unit 197 is configured to provide that signal to a remote processing location via the Internet or the like, where the signal was evaluated. Upon an evaluation that an action should be taken or otherwise can be utilitarian taken by the sleep apnea system 1991, the unit 197 activates to implement sleep apnea countermeasures, which countermeasures are conducted by a hose 1902 sleep apnea mask 195. By way of example only and not by way of limitation, pressure variations can be used to treat the sleep apnea upon an indication of such an occurrence.

In an exemplary embodiment, the advanced implantation methods and devices detailed herein can be utilized to treat sleep apnea. Specifically, the electrodes of the implant disclosed below can be utilized in place of the electrodes 194 (placed accordingly, of course), and the implant can be of a configuration to treat sleep apnea. In this regard, in an exemplary embodiment, the implantable components detailed herein can be located at locations to treat sleep apnea in accordance with the teachings herein, with the requisite modification if necessary or otherwise utilitarian to implement such.

FIGS. 1C and 1D provide another exemplary schematic of another exemplary conceptual sleep apnea system 1992. Here, the sleep apnea system is different from that of FIG. 1B in that electrodes 194 (which can be implanted in some embodiments) are utilized to provide stimulation to the human who is experiencing a sleep apnea scenario. FIG. 1C illustrates an external unit, and FIG. 1D illustrates the external unit 120 and an implanted unit 110 in signal communication via an inductance coil 707 of the external unit and a corresponding implanted inductance coil (not shown) of the implanted unit, according to which the teachings herein can be applicable. Implanted unit 110, can be configured for implantation in a recipient, in a location that permits it to modulate nerves of the recipient 100 via electrodes 194. In treating sleep apnea, implant unit 110 and/or the electrodes thereof can be located on a genioglossus muscle of a patient. Such a location is suitable for modulation of the hypoglossal nerve, branches of which run inside the genioglossus muscle.

External unit 120 can be configured for location external to a patient, either directly contacting, or close to the skin of the recipient. External unit 120 may be configured to be affixed to the patient, for example, by adhering to the skin of the patient, or through a band or other device configured to hold external unit 120 in place. Adherence to the skin of external unit 120 may occur such that it is in the vicinity of the location of implant unit 110 so that, for example, the external unit 120 can be in signal communication with the implant unit 110 as conceptually shown, which communication can be via an inductive link or an RF link or any link that can enable treatment of sleep apnea using the implant unit and the external unit. External unit 120 can include a processor unit 198 that is configured to control the stimulation executed by the implant unit 110. In this regard, processor unit 198 can be in signal communication with microphone 12, via electrical leads, such as in an arrangement where the external unit 120 is a modularized component, or via a wireless system, such as conceptually represented in FIG. 1D.

A common feature of both of these sleep apnea treatment systems is the utilization of the microphone to capture sound, and the utilization of that captured sound to implement one or more features of the sleep apnea system. In some embodiments, the teachings herein are used with the sleep apnea device just detailed.

Returning back to hearing prosthesis devices, and in particular a cochlear implant, FIG. 1E is a side view of the internal component of cochlear implant 100 without the other components of system 10 (e.g., the external components). Cochlear implant 100 comprises a receiver/stimulator 180 (combination of main implantable component 120 and internal energy transfer assembly 132) and a stimulating assembly or lead 118. Stimulating assembly 118 includes a helix region 182, a transition region 184, a proximal region 186, and an intra-cochlear region 188. Proximal region 186 and intra-cochlear region 188 form an electrode array assembly 190. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the electrode array assembly 190. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected through lead 118 to receiver/stimulator 180, through which respective stimulating electrical signals for each electrode contact 148 travel.

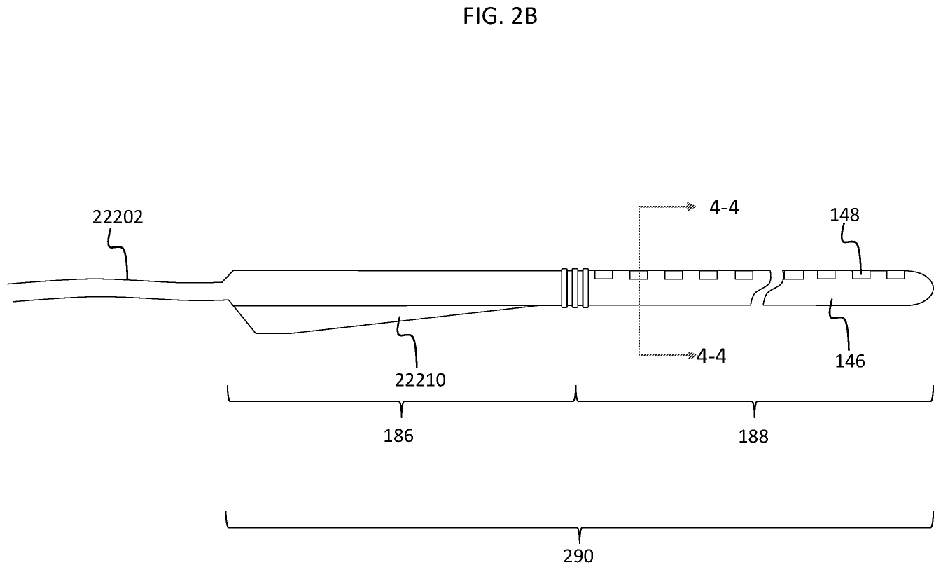

FIG. 2A is a side view of electrode array assembly 190 in a curled orientation, as it would be when inserted in a recipient's cochlea, with electrode contacts 148 located on the inside of the curve. FIG. 2A depicts the electrode array of FIG. 1B in situ in a patient's cochlea 140.

FIG. 2B depicts a side view of a device 290 corresponding to a cochlear implant electrode array assembly that can include some or all of the features of electrode array assembly 190 of FIG. 1B. More specifically, in an exemplary embodiment, stimulating assembly 118 includes electrode array assembly 290 instead of electrode array assembly 190 (i.e., 190 is replaced with 290).

Electrode array assembly 290 includes a cochlear implant electrode array componentry of the 190 assembly above. Note also element 22210, which is a quasi-handle like device utilized with utilitarian value vis-à-vis inserting the 188 section into a cochlea. By way of example only and not by way of limitation, element 22210, which is a silicone body that extends laterally away from the longitudinal axis of the electrode array assembly 290, and has a thickness that is less than that of the main body of the assembly (the portion through which the electrical leads that extend to the electrodes extend to the elongate lead assembly 22202). The thickness combined with the material structure is sufficient so that the handle can be gripped at least by a tweezers or the like during implantation and by application of a force on to the tweezers, the force can be transferred into the electrode array assembly 290 so that section 188 can be inserted into the cochlea.

FIG. 2C presents additional details of an external component assembly 242, corresponding to external component 142 above.

External assembly 242 typically comprises a sound transducer 220 for detecting sound, and for generating an electrical audio signal, typically an analog audio signal. In this illustrative arrangement, sound transducer 220 is a microphone. In alternative arrangements, sound transducer 220 can be any device now or later developed that can detect sound and generate electrical signals representative of such sound. An exemplary alternate location of sound transducer 220 will be detailed below. As will be detailed below, a sound transducer can also be located in an ear piece, which can utilize the “funneling” features of the pinna for more natural sound capture (more on this below).

External assembly 242 also comprises a signal processing unit, a power source (not shown), and an external transmitter unit. External transmitter unit 206 (sometimes herein referred to as a headpiece) comprises an external coil 208 and, a magnet (not shown) secured directly or indirectly to the external coil 208. The signal processing unit processes the output of microphone 220 that is positioned, in the depicted arrangement, by outer ear 201 of the recipient. The signal processing unit generates coded signals using a signal processing apparatus (sometimes referred to herein as a sound processing apparatus), which can be circuitry (often a chip) configured to process received signals—because element 230 contains this circuitry, the entire component 230 is often called a sound processing unit or a signal processing unit. These coded signals can be referred to herein as a stimulation data signals, which are provided to external transmitter unit 206 via a cable 247. In this exemplary arrangement of FIG. 1D, cable 247 includes connector jack 221 which is bayonet fitted into receptacle 219 of the signal processing unit 230 (an opening is present in the dorsal spine, which receives the bayonet connector, in which includes electrical contacts to place the external transmitter unit into signal communication with the signal processor 230). It is also noted that in alternative arrangements, the external transmitter unit is hardwired to the signal processor subassembly 230. That is, cable 247 is in signal communication via hardwiring, with the signal processor subassembly. (The device of course could be disassembled, but that is different than the arrangement shown in FIG. 1D that utilizes the bayonet connector.) Conversely, in some embodiments, there is no cable 247. Instead, there is a wireless transmitter and/or transceiver in the housing of component 230 and/or attached to the housing (e.g., a transmitter/transceiver can be attached to the receptacle 219) and the headpiece can include a receiver and/or transceiver, and can be in signal communication with the transmittertransceiver of/associated with element 230.

FIG. 1F provides additional details of an exemplary in-the-ear (ITE) component 250. The overall component containing the signal processing unit is, in this illustration, constructed and arranged so that it can fit behind outer ear 201 in a BTE (behind-the-ear) configuration, but may also be worn on different parts of the recipient's body or clothing.

In some arrangements, the signal processor (also referred to as the sound processor) may produce electrical stimulations alone, without generation of any acoustic stimulation beyond those that naturally enter the ear. While in still further arrangements, two signal processors may be used. One signal processor is used for generating electrical stimulations in conjunction with a second speech processor used for producing acoustic stimulations.

As shown in FIG. 1F, an ITE component 250 is connected to the spine of the BTE (a general term used to describe the part to which the battery 270 attaches, which contains the signal (sound) processor and supports various components, such as the microphone—more on this below) through cable 252 (and thus connected to the sound processor/signal processor thereby). ITE component 250 includes a housing 256, which can be a molding shaped to the recipient. Inside ITE component 250 there is provided a sound transducer 220 that can be located on element 250 so that the natural wonders of the human ear can be utilized to funnel sound in a more natural manner to the sound transducer of the external component. In an exemplary arrangement, sound transducer 242 is in signal communication with remainder of the BTE unit via cable 252, as is schematically depicted in FIG. 1F via the sub cable extending from sound transducer 242 to cable 252. Shown in dashed lines are leads 21324 that extend from transducer 220 to cable 252. Not shown is an air vent that extends from the left side of the housing 256 to the right side of the housing (at or near the tip on the right side) to balance air pressure “behind” the housing 256 and the ambient atmosphere when the housing 256 is in an ear canal.

Also, FIG. 1D shows a removable power component 270 (sometimes battery back, or battery for short) directly attached to the base of the body/spine 230 of the BTE device. As seen, the BTE device in some embodiments includes control buttons 274. The BTE device may have an indicator light 276 on the earhook to indicate operational status of signal processor. Examples of status indications include a flicker when receiving incoming sounds, low rate flashing when power source is low or high rate flashing for other problems.

In one arrangement, external coil 130 transmits electrical signals to the internal coil via an inductance communication link. The internal coil is typically a wire antenna coil comprised of at least one, or two or three or more turns of electrically insulated single-strand or multi-strand platinum or gold wire. The electrical insulation of the internal coil is provided by a flexible silicone molding (not shown). In use, internal receiver unit may be positioned in a recess of the temporal bone adjacent to outer ear 101 of the recipient.

With the above as a primer (the above should be considered base technologies from which we build upon, and are not part of the invention, but the teachings below can use any one or more of these features in some embodiments, providing that the art enables such), embodiments are directed to cochlear implants and middle ear implants and DACS that, in some embodiments, utilize one or more of the teachings above, albeit modified in at least some instances, to practice the teachings herein.

Also, while the teachings associated above are typically directed towards a cochlear implant, the disclosure of such and any teachings herein relating to such also correspond to a disclosure of an implantable/implanted device that is a middle ear implant or a DACS, that utilizes some of the pertinent teachings (e.g., both will utilize the inductance communication for power, for example). The output will be different (mechanical stimulation vs. electricity), and thus the “stimulator” features will also be different, as is understood in the art.

Cochlear implant electrodes are expected to deliver stimulation over the lifetime of the recipient (implantee in this case), potentially, 30, 50, or 75 years or more. Wear of one or more of the electrodes can occur over these time periods (or shorter, in some instances—more on this below), resulting in eventual reduction of utility/reduction of function or loss of utility/function. The smaller the electrode size in general, and the surface area exposed to the ambient environment in particular, the increased rate of passive dissolution/active dissolution, or at least the sooner the electrode will experience of the solution level that deleteriously affects functionality. In at least some exemplary embodiments of the cochlear implant electrode utilized with the teachings detailed herein, an increase in charge density of the electrode results in the increased dissolution rate and/or the shortened utilitarian life expectancy of the electrode/the faster the electrode array reaches the end of its useful life.

And while embodiments depicted above present a 22 electrode cochlear implant electrode array, embodiments include arrays that include 30, 35, 40, 45, 50, 60, 70, 80, 90, 100, 120, 150, 175, 200, 300, 400, 500, 600, or 700 or more electrodes, or any value or range of values therebetween in 1 increments (e.g., 55, 62, 33 to 77, etc.). In an exemplary embodiment, these numbers are located within 1, 1.5, 2, 2.5, or 3 inches or any value or range of values therebetween in 0.1 inch increments of each other and/or arrayed in a linear or substantially linear fashion that extends the aforementioned distance. In an exemplary embodiment, the aforementioned numbers of electrodes are located within a 0.5, 0.75, 1, 1.25, 1.5, 1.75, 2, 2.5, 3, 3.5, 4, 4.5, or 5 square inch, or any value or value or range of values therebetween in 0.1 in.² increments. Increased electrode density (number of electrodes per a spatial unit) can have utilitarian value with respect to increasing the spectral resolution of a delivered signal (or a sensed phenomenon—as will be detailed below, embodiments detailed herein are not only directed towards devices that provide stimulation to tissue, but are also applicable to devices that sense phenomenon within a human being, such as by way of example only and not by way of limitation, the electrodes of the pacemaker). Thus, embodiments include increasing the spectral resolution of a delivered signal by increasing the number of electrodes within a given spatial dimension. This results in an increased charge density relative to that which would otherwise be the case with a lower number of electrodes in the same given area (where, for example, the working surface of the electrode can thus be larger, because there is more room).

At least some exemplary embodiments according to the teachings detailed herein apply multipolar stimulation from the electrodes of the implant. This can result in higher charge levels for a given electrode relative to that which would be the case with respect to utilizing those same electrodes for monopolar stimulation. Multipolar stimulation as used in at least some exemplary embodiments herein can have utilitarian value with respect to focusing the stimulation and improving hearing performance, relative to the utilization of monopolar stimulation for example, and can also improve channel independence, and spectral resolution and speech understanding, all relative to that which would be the case in the instance of the utilization of monopolar stimulation. Still, in embodiments that utilize the electrode arrays according to the teachings detailed herein, this can further add to the statistical likelihood and/or actual occurrence of premature electrode dissolution (relative to that which will be the case utilizing monopolar stimulation—all else being equal (all comparisons herein are with regard to all else being equal unless otherwise noted).

It is noted that in some embodiments, charged balanced biphasic waveforms and use of inert materials such as platinum are utilized to prolong the longevity of the electrodes relative to that which would otherwise be the case.

The rate of dissolution for a particular electrode in at least some exemplary scenarios of the embodiments detailed herein is known, or at least educatedly suspected, to depend on at least one or more factors of factors, such as by way of example only and not by way of limitation, stimulation waveform amplitude and/or pulse width, the number of pulses delivered per hour/day/week, month, etc., position on the carrier of the electrodes, position on the array, position in the body (e.g., position in the cochlea), degree of fibrous tissue growth (relative to zero fibrous tissue growth) and/or chemical composition of the ambient environment of the electrode (e.g., in case of a cochlear implant electrode array, the perilymph inside the cochlea).

In models to which the teachings detailed herein can be applicable, 45% of the variance of the rate of dissolution is not accounted for by charge. Thus, a simple apriori prediction for each individual person or electrode is not feasible in at least some exemplary scenarios of use of the various electrodes/medical devices disclosed herein and other applicable devices. Therefore, whatever measures that are included in a system design to maximize the desired lifetime of the implant, there will remain distribution of dissolution rates that can only be poorly estimated at the time of development/implantation. There will typically be, in at least some exemplary scenarios, a residual risk for each individual recipient that their respective particular dissolution rate will be at the high end of the distribution (or otherwise at a higher end relative to a mean/median and/or mode, for example), resulting in unexpected/unpredicted and premature wear out of their particular implant, potentially necessitating replacement.

It is briefly noted that the phrases “dissolution,” “erosion” and “wear” will be variously used herein to describe phenomenon associated with electrodes. These refer to a change in the state of the electrode itself relative to that which was the case when the electrode was new. This as distinguished from, for example, a film or the like that forms on the electrode surface. That does not change the state of the electrode itself—that is a change to an environment of the electrode that may or may not impact the performance of the electrode. Passive dissolution as used herein refers to the reduction in the amount of material of the electrode owing to chemical reactions due to the environment without electrical current application. Active dissolution as used herein refers to the reduction in the amount of material of the electrode due to use of the electrode (to provide electrical current to the environment). More specifically, the removal of material from the electrode occurs due to (electro)chemical reactions that occur when an electrode is at rest potential (i.e., not used to pass current to the environment) in an electrolyte. Removal of material also occurs due to electrochemical reactions, often at a faster rate, when an electrode is used to provide electrical current to the environment. It's the same electrochemical process, just modified by the changing potential on the electrode that occurs during electrical stimulation. Thus, passive dissolution corresponds to the reduction of material that occurs electrochemically when an electrode sits at rest in an electrolyte, and active dissolution is the process that occurs to remove electrode material when the electrode is used to pass current.

Wear as used herein covers both of these phenomena (as well as erosion), as well as any other phenomenon that may reduce the amount of material of a given electrode over time. Any disclosure of passive dissolution and/or active dissolution corresponds to a disclosure of wear, where the wear is the combination of the two if it is the “and.” For example, if there is a disclosure of determining a passive dissolution rate and/or (or just “and”) an active dissolution rate, that corresponds to determining a wear rate. That is, this corresponds to the combination of the active and passive dissolution (save for the “or,” which is of course is one or the other, and thus the wear is due to the active dissolution or the passive dissolution).

Sometimes, one of these phrases will be used in the absence of another of these words. Unless otherwise noted, any statement herein that utilizes one of these phrases corresponds to a disclosure where such is the case with respect to another one of these phrases, providing that such is technically correct. This is done in the interest of textual economy. This does not mean that they both mean the same thing. This is only to show that the disclosure of one can correspond to a disclosure of an alternate embodiment of the other even though the specific words are not typed onto the page, again in the interest of textual economy.

Dissolution covers both active and passive dissolution, but it is noted that any disclosure herein of dissolution also corresponds to a disclosure of both of the species separately for the purposes of textual economy. This is also the case with wear—a disclosure of wear corresponds to a disclosure of both species of dissolution.

Some embodiments of the teachings detailed herein can include devices systems and/or methods that can enable the detection of wear (passive dissolution and/or active dissolution, etc.) and/or otherwise provide an estimation of the status of one or more implanted electrodes with respect to wear beyond that which would be the case with respect to requiring the explanation of the electrodes. That is, embodiments can provide the detection of wear and/or the estimation of wear or otherwise provide an estimation of the remaining lifetime of one or more electrodes implanted in the human body without having to remove the electrodes and analyze the electrodes or otherwise access the electrodes via a surgical/invasive procedure (beyond that which resulted in the electrodes being implanted, of course).

Some embodiments of the teachings detailed herein provide the aforementioned detection and/or estimation at a level that is not possible with the current state of technology on Mar. 20, 2021, as would be approved by the FDA in the United States of America on that date, and/or as would be approved by the pertinent regulatory authority in the United Kingdom, the Commonwealth of Australia, New Zealand, Canada, the Republic of France, and/or the Federal Republic of Germany, and/or the People's Republic of China.

Some embodiments of the teachings herein provide for the avoidance of a deleterious wear event of a given electrode (e.g., it no longer can stimulate at a utilitarian level) over the electrodes lifetime or otherwise extend the lifetime of the electrode beyond that which would otherwise be the case. By way of example only and not by way of limitation, by implementing any one or more of the teachings detailed herein, and electrode(s) functional life while implanted can be extended greater than 1.25, 1.5, 1.75, 2, 2.25, 2.5, 2.75, 3, 3.5, 4, 4.5, 5, 5.5, 6, 7, 8, 9, or 10 times or longer, or any amount or range of amounts therebetween in 0.05 increments beyond that which would otherwise be the case in the absence of the implementations of one or more of the teachings detailed herein. Thus, embodiments of the teachings detailed herein provide for the determination of a deleterious electrode wear event in progress or that will occur so that utilitarian actions can be taken to remediate or otherwise avoid that event.

A utilitarian result of the teachings detailed herein is that one or more stimulation channels of a cochlear implant, by way of example, can be continued to be utilized beyond that which would otherwise be the case if the deleterious wear event were to occur (the maintenance can be for any one or more of the temporal periods noted above with respect to the electrode). Further, the teachings detailed herein can be utilized to determine why certain channels will no longer provide utilitarian stimulation (and in some embodiments that the channels are no longer providing utilitarian stimulation), thus enabling workaround channels to be developed by adjusting the electrodes that are utilized to provide stimulation, where electrodes that still have structural effectivity for stimulation can be utilized to a degree greater than that which would otherwise be the case so as to compensate for the now defunct electrodes of the underlying initial channels.

In an exemplary embodiment, there is a medical device that includes electrodes, such as, for example, a cochlear implant, a sleep apnea device that utilizes implanted electrodes, a retinal prosthesis, a spine stimulator, a pacemaker, and epilepsy monitoring device, and epilepsy treatment device (that uses electrodes to provide stimulation to the brain with the nerves), a vagus nerve stimulator, an EKG monitor, an EEG monitor, a heart stimulator, etc. In this exemplary embodiment, the medical device includes an implantable portion of the medical device, the implantable portion including at least one electrode, including a plurality of electrodes, including any one or more of the numbers of electrodes detailed above. Further, the implantable portion is configured to, while implanted in a human, obtain data indicative of wear and/or passive dissolution and/or active dissolution of at least one of the electrodes of the plurality of electrodes.

At least some of the teachings detailed herein, as can be inferred, are applicable to active, as opposed to passive, implantable medical devices, such as by way of example only and not by way of limitation, pain stimulators, vestibular stimulators, deep brain stimulators, cardiac devices, etc. other embodiments are directed towards passive implantable medical devices.

In an exemplary embodiment, the implantable portion is the internal component(s) of FIG. 1E. The electrodes can be the electrodes 148 of FIG. 2A and/or FIG. 2B. In an exemplary embodiment, the implantable portion corresponds to a receiver-stimulator of the cochlear implant. In an exemplary embodiment, the receiver stimulator can have a logic circuit that can be configured to control the application of electrical signals to the various pertinent electrode(s) so as to provide voltage differentials in a controlled manner between electrodes. Alternatively, and/or in addition to this, the logic circuit can be in the external component of the cochlear implant, and this logic circuit can control the application of the pertinent electrical signals to the pertinent electrodes. Further, the implantable component can be configured to provide a telemetry signal to the external device indicative of voltage readings and/or current readings, etc., from the electrodes. In an exemplary embodiment, the telemetry signal can be the raw data, while in other embodiments, the telemetry signal can be a signal that is indicative of the results of an analysis that is executed by the implantable component.

In an exemplary embodiment, the cochlear implant can correspond to or otherwise be based on or otherwise be a modified version of the cochlear implant described in US patent application publication number 2012/0316454, published Dec. 13, 2012, entitled Electrode Impedance Spectroscopy, to the pioneer in impedance evaluation of cochlear implants, Paul Carter. In this regard, the device of the '454 publication can be utilized or otherwise modified so that it can be utilized to obtain data indicative of the passive dissolution and/or active dissolution of the at least one electrode. Other devices/methods that can be used or otherwise modified to obtain the data noted above are indicative of passive dissolution and/or motion or otherwise wear of the one or more electrodes are disclosed in the following patent application publications:

WO2018/173010, published Sep. 27, 2018, entitled Advanced Electrode Array Location Evaluation, to Inventor Nicholas Pawsey;

WO 2019/162837, entitled Advanced Electrode Data Analysis, published Aug. 29, 2019, to Inventor Paul Carter; and

WO 2019/175764, published September 2019, entitled Electrical Field Usage in Cochleas, to Inventor Ryan Melman.

The above noted patent application publications disclose devices and methods that can be utilized or otherwise modified to obtain data relating to the various electrodes. Additional details of such will be described below, but it is noted that these various teachings utilize the existing electrodes of a cochlear implant electrode array, in combination with the electronics of the implant and/or the external component, to obtain electrical measurements relating to the electrodes. The signals applied to the electrodes can be modified to provide the stimulus that results in enablement of a phenomenon that can be read by read electrodes that corresponds to the obtained data. The point here is that the structure and the methods disclosed in those applications can be modified accordingly to implement the teachings detailed herein.

In an exemplary embodiment, consistent with the above-noted discussion regarding the telemetric features of at least some of the apparatuses disclose in the above noted publications, the implantable portion is configured to communicate the obtained data and/or data based on the obtained data transcutaneously to a device located outside the human. In an exemplary embodiment, this is achieved via the receiver stimulator, which includes an inductance coil, where the transceiver of the receiver stimulator is configured to provide a telemetric signal from the implant, through the skin of the human, to the external device (see FIG. 1 for example). This is the opposite of how the device normally works, where the external component captures sound, converts that sound into electrical signal that is applied to the inductance coil and the external component, which is transcutaneously communicated via an inductance link to the implant.

In some embodiments, the medical device is configured to analyze the obtained data and determine a wear status of the at least one electrode and communicate an indication of the wear status. In an exemplary embodiment, the wear status can be specifically a passive dissolution status and/or any active dissolution status of the electrode. In an exemplary embodiment, the analysis is executed by the external component, which can be in the form of a microprocessor or otherwise electronic circuitry with logic circuits configured to analyze the data and extract indicators (which will correspond to latent variables in at least some embodiments, again, more details of this below) that can be utilized to deduce the wear status of the electrode(s). The communication of the wear status can be by way of a USB port on the external component or by way of a Bluetooth link with a remote device, or any other telemetric arrangement that can have utilitarian value with respect to communicating the wear status. The wear status can be stored in a memory, and this memory could be periodically accessed.

It is also noted that in some embodiments, it is the implantable portion that is configured to analyze the obtained data and determine a wear status of the at least one electrode and communicate an indication of the wear status. The pertinent electronics microprocessors can be located in the receiver-stimulator or another component of the implant. The data signal can be sent with the telemetry link noted above.

In some exemplary embodiments, the medical device is configured to enable an adjustment of an operation of the cochlear implant to reduce a future passive dissolution and/or active dissolution rate of the at least one electrode and/or one or more other electrodes of the implantable component. Additional details of this will be described below, but by way of example only and not by way of limitation, in an exemplary embodiment, the medical device can be configured to enable the reduction of a pulse rate and/or a degree of focusing for some or all stimulation channels to reduce a stimulation amplitude associated with a given electrode relative to that which was otherwise the case before the adjustment.

It is also noted that in an exemplary embodiment, the medical device can be configured to analyze the obtained data and determine a wear status of the at least one electrode. That is, instead of simply being able to obtain the data indicative of wear of the at least one electrode, the medical device can be configured to actually use that obtain data for the utilitarian purpose just noted. This can be in combination with the device that is configured to enable the adjustment, or, in other embodiments, this can simply be a standalone feature that simply provides a warning or otherwise provides an indication that the electrode(s) (any reference to an electrode corresponds to a disclosure of an alternate embodiment of two or more electrodes or all of the electrodes of the medical device and was otherwise noted) is experiencing a passive dissolution and/or an active dissolution phenomenon that could be problematic in the short and/or long term. Indeed, in an exemplary embodiment, the wear status can simply be that there is a deleterious event that is occurring or otherwise will occur that will ultimately result in a potential problem. This as opposed to, for example, other embodiments, where the wear status is a percentage range of the electrode that remains for example (or a ballpark). Accordingly, such a specific wear status would be just that, a specific wear status. A wear status is a genus that encompasses the species of a specific wear status, which includes data that would enable one of ordinary skill in the art to reduce an approximate physical state of the electrode and/or deduce or otherwise estimate the remaining life of the electrode. Put another way, a wear status would be analogous to an indication that a tire pressure is low, and a specific wear status would be analogous to an indication that the tires are at between 65 and 75% of the pressure that they otherwise should be, etc.

All the above said, in an exemplary embodiment, the medical device is a relatively sophisticated device, which device is configured to analyze the obtained data and determine a specific wear status of the at least one electrode and automatically adjust an operation of the cochlear implant to reduce a future passive dissolution and/or active dissolution rate of the at least one electrode and/or one or more other electrodes of the implantable component. Again, as promised above, the adjustments that can be made will be detailed below. We further note that here, the device determines the specific wear status, as distinguished from the broader concept of the wear status. Also, we note that in this exemplary embodiment, the adjustment can not only extend the longevity or otherwise reduce the future passive dissolution and/or active dissolution rate of one electrode, such as the electrode from which the data is obtained, but can also extend the longevity or otherwise reduce the future passive dissolution and/or active dissolution rate of another electrode. In this regard, one or more of the electrodes can be utilized as test electrodes (they can also be fully functioning stimulating electrodes), which can be utilized as a proxy for the status of other electrodes. Further, in an exemplary embodiment, the device could make a determination that the electrode upon which the data is related is a “hopeless electrode,” and thus could make determinations to preserve the other electrodes. Thus, embodiments can include a medical device that is configured to analyze the obtained data and determine a wear status (which can include in some embodiments a specific wear status—wear status is a genus—a general wear status would exclude a specific sear status) of the at least one electrode and automatically adjust an operation of the cochlear implant to compensate for the wear of the at least one electrode. By way of example only and not by way of limitation, this could include shifting channels or implementing a constructive and/or destructive interference regime, such as that disclosed in US Patent Application publication 2010/0198301 to Zachary Smith, published Aug. 5, 2010, entitled Multi-electrode Channel Configurations, and/or U.S. Pat. No. 7,860,573 to Christopher van den Honert, published Dec. 28, 2010, entitled Focused Stimulation in a Medical Stimulation Device. Indeed, to be clear, in some embodiments, the unadjusted device operates according to one or more of the teachings of these two publications. It is that these publications enable the adjustments of the focusing of the stimulation in a manner that can account for the electrode that has suffered a deleterious event.

Of course, embodiments can be directed towards reducing the pertinent rates of both the electrode upon which the obtained data is based and one or more other electrodes.

In some embodiments, the medical device is configured to analyze the obtained data and determine a wear status of the at least one electrode and automatically recommend an action to reduce a future passive dissolution and/or active dissolution rate of the at least one electrode and/or one or more other electrodes of the implantable component and/or recommend an adjustment to an operation of the cochlear implant to compensate for the wear of the at least one electrode. With respect to the last feature, this was just described except with respect to the automatic adjustment. Here, the device simply recommends the adjustment. It is up to the user or a healthcare professional or a technician to implement the adjustment (which could be as simple as accepting the recommended adjustment). With respect to the former feature, this too has been discussed above, except with respect to automatic action to reduce the future passive dissolution and/or active dissolution rate. But to be clear, some embodiments can also utilize the determination and/or recommendation and/or analyses detailed herein to implement automatic action that alters stimulation parameters and/or the operation of the hearing prostheses, which automation can be executed by a software algorithm or any other computational regime that can enable such. This can be done automatically by the medical device and/or by a component in signal communication with the medical device, such as a handheld smart phone or the like, etc.

It is noted that while the above has been directed towards features of the medical device, it is noted that some of these features can exist alternatively and/or in addition to this in an external remote device that is remote from the medical device, such as by way of example only and not by way of limitation, a smart phone or smart device that is in signal communication with the medical device, such as by way of example only and not by way of limitation by a Bluetooth connection, or a computer such as a laptop or desktop computer that can be placed into signal communication with the medical device, and/or a device that is accessible via the Internet or the like which is located at or otherwise is embodied in a remote server that is tens of miles or more from the medical device. This is pertinent to the diagnostic and/or recommendation features detailed above. By way of example only and not by way of limitation, the actions of the analysis of the obtained data and the determination of the wear status and/or the recommended adjustment in operation, etc., can be executed by a remote computing device, such as a trained neural network by way of example, or any other computing device that can enable the teachings detailed herein. This data can be communicated to a healthcare professional for final approval, or can be communicated back to the user or the technician in control of the medical device. In an exemplary embodiment, as can be communicated directly to the medical device for appropriate implementation.

In at least some exemplary embodiments, the various measurements or otherwise data collection techniques detailed herein and/or variations thereof that have utilitarian value to implement the teachings detailed herein can be made on each electrode contact and/or a selection of contacts or otherwise a representative contact at regular intervals (daily, weekly, monthly, etc.) and/or irregular interviews and logged (e.g., by the implantable portion and/or the external portion of the prostheses and/or can be collected via a smartphone and/or a personal computer, etc.). This data can be provided to a data collection center or a data evaluation center in real time or at a later date. Changes in one of more of these measurements over time in a way that is known to be characteristic of active dissolution and/or passive dissolution of electrical contacts can be detected by a software algorithm running in the implantable portion and/or on the external portion of the prostheses processor and/or in the aforementioned smart phone and/or PC, and/or at a remote data analysis center, which can be at a remote server as noted above. The algorithm may also consider data that is logged relating to the usage of the cochlear implant, such as, for example, the number of stimulation pulses and/or the current amplitude and/or the phase duration of pulses delivered over the life of the device through one or more or all contacts. Any operational parameter that can be logged that can be utilized to estimate or otherwise deduce the current wear state and/or a wear rate of an electrode can be utilized in at least some exemplary embodiments. The algorithm may also take into account the position of the contact on the array, the position of the array within the human, such as, for example, the position of a cochlear implant electrode array within a cochlear (which may be established though analysis of post-op x-ray and/or CAT scan), and/or the status of neighboring electrodes contacts. These spatially based features can impact the various measurements detailed herein. By taking into account the spatial variables, the accuracy of the data analysis can be further improved so as to further increase the accuracy of the analysis relating to the current wear rate and/or the current wear status of the electrode. The algorithm can then calculate an estimate of the wear status and/or wear rate of one or more or all of the electrode contacts on the array. This information can be delivered to a clinician who can decide whether changes to the prostheses, such as changes to stimulation parameters associated with a cochlear implant, are warranted in order to reduce the risk of premature wear out and/or otherwise to determine how to accommodate or otherwise address the fact that an electrode has effectively worn out. Alternatively, human evaluation can be utilized to evaluate the estimate of the wear status and/or wear rate of one or more or all of the electrode contacts of the array.

Any of the diagnostic and/or the determination and/or recommendation and/or analysis and/or adjustments detailed herein that are executed by the medical device can be executed by a laptop computer and/or desktop computer and/or a smart phone or smart device that is handheld and/or by server that is remote from the medical device accessible via the Internet was otherwise noted providing that the art enable such. It is also noted that in some embodiments, any of the diagnostic and/or determination and/or recommendation and/or analysis and/or adjustments detailed herein can be executed by a trained professional such as a healthcare professional or a technician unless otherwise noted providing that the art enable such.

As briefly noted above, and as will be described in greater detail below, latent variables for example can be utilized to ascertain or otherwise estimate a passive dissolution state and/or active dissolution state of an electrode (and thus a wear state of the electrode). In an exemplary embodiment, electrical properties such as voltage and/or current and/or impedance that can be measured by the implanted device can be impacted by properties of an electrode at different points during its lifetime. These properties can be measured by executing and impedance measurement for example within a cochlear implant. This can be executed by passing a measurement current between two electrodes, such as, for example, electrode 6 out of the 22 electrodes of an electrode array and an extra cochlear electrode—in an alternative embodiment, one of the electrodes of the array can be a source, and another can be a sink instead of using the extra cochlear electrode). Voltage can be measured between the same electrodes (electrode 6 and the extra cochlear electrode, in this example). The aforementioned voltage can be measured at some time while the current is being passed. The impedance can be calculated and/or otherwise derived by, for example, dividing the voltage by the current. Any disclosure herein of measuring or otherwise ascertaining impedance corresponds to a disclosure where, for example, this aforementioned technique is utilized. Any method of measuring and/or obtaining electrical properties that can enable the teachings herein can be used in some embodiments, providing that such is safe for the recipient, unless otherwise indicated.

The properties that are measured that can enable the ascertention of wear can be measured by implementing the teachings and/or modified teachings of the above noted patent applications by Carter and/or Pawsey and/or Melman. Accordingly, there is utilitarian value with respect to providing an electrode array in general, and an electrode in particular, the results in a relatively distinct change in one or more of the aforementioned electrical properties upon a given passive dissolution and/or active dissolution status. Accordingly, in an exemplary embodiment, there is an electrode array, such as a cochlear implant electrode array, or any other device that utilizes electrodes such as those detailed herein or others to which the teachings herein are applicable, comprising a plurality of electrodes, a carrier carrying the electrodes. In this embodiment the implantable electrode array is configured to enable in vivo analysis of a passive dissolution status and/or active dissolution status of at least one electrode of the plurality of electrodes. In an exemplary embodiment, the implantable electrode array is configured to provide an abrupt change in an electrical phenomenon upon one or more electrodes reaching a passive dissolution status and/or active dissolution status. In an exemplary embodiment, the implantable electrode array is configured to provide an a change of at least 10, 20, 30, 40, 50, 60, or 70%, or any value or range of values therebetween in 1% increments of an electrical phenomenon (where the denominator is the phenomenon before the change) upon one or more electrodes reaching a passive dissolution status and/or active dissolution status. In an exemplary embodiment, this can be achieved by using a composite electrode, wherein the composite nature enables the in vivo analysis.

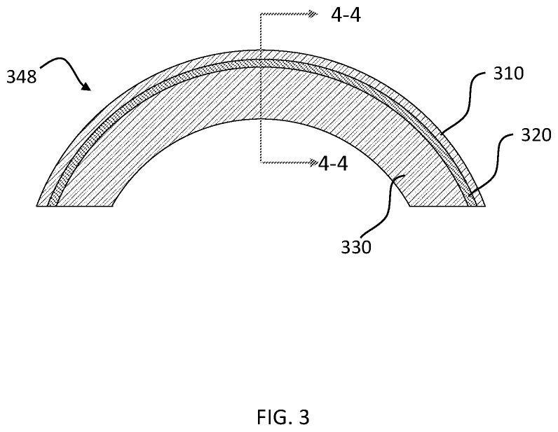

More specifically, seen in FIG. 3 is a side view of a composite electrode 348 when viewed looking down the longitudinal axis of the intracochlear section 188 of the electrode array of FIG. 2B. Electrode 348 is a so called half band electrode. It is noted that the teachings detailed herein can be applicable to other types of electrodes, such as a full band electrode and/or a planar electrode, etc. Also, while the surface areas of the electrodes implicated in the embodiment of FIG. 3 constituted generally rectangular surface area exposed to the ambient environment (at least when superimposed onto a plane of view), in other embodiments, the surface area can be circular or oblong, etc. Here, there is a top layer 310 which is exposed to the ambient environment of the electrode array (e.g., the cochlea, when implanted in the cochlea). This layer is located above a second layer 320, which in turn is located above a third layer 330. In this exemplary embodiment, layers 310 and 330 are made of platinum or platinum alloy, and layer 320 is made of iridium. That said, in an alternative embodiment, layers 310 and 330 can be made of iridium and layer 320 can be made of platinum. Moreover, in an alternate embodiment, there can be only two layers for example, where the top layer is iridium and the bottom layer is platinum, wherein the top layer corresponds to a thickness of layer 310 and the bottom layer corresponds to a combined thickness of layers 320 and 330 as represented in FIG. 3 (but there are only two layers). Other materials, such as gold or silver or any other material that can have utilitarian value with respect to an electrode array can be utilized providing that such enable the teachings detailed herein.

And what of those teachings. Here, in this exemplary embodiment, the layers are such that they have a different electrical property relative to one another, all other things being equal. The electrical properties are monitored in some embodiments, daily, weekly, monthly, yearly, etc., and upon a change of the electrical properties or otherwise upon an indication that the electrical property is indicative of one of the lower layers, certain features regarding the wear of the electrode can be deduced.

More specifically, in an exemplary embodiment, using additive manufacture, a different material can be incorporated into the electrode contact at a certain depth to establish the point at which the electrode has dissolved to that point. As noted above, the different material can be iridium, or some other material that, in some embodiments, behaves very similarly to the other material (which can be platinum), while in other embodiments the behavior could be different. In an exemplary manner, the material can be material that was otherwise has been and has been used as an electrode material (or more specifically, an implanted electrode material). In an exemplary embodiment, the ratio in totality on a per unit volume and/or a per unit mass of the electrode can be 70/30, 75/25, 80/20, 85/15, 90/10, 95/5, 98/2 or 99/1 or even 99.9/0.1 or any value or range of values therebetween in 0.01 increments on the numerator and/or the denominator, where the smaller amount can be, for example, iridium, and the larger amount can be platinum.

In at least some exemplary embodiments, the different material utilized (the minority material, here, iridium) can have a different cyclic voltammetry (CV) spectrum relative to the mass majority material (here, platinum). Accordingly, in some exemplary embodiments, periodic CVs or non/periodic CVs are performed using the implantable component in part or in total (the implantable component can be a totally implantable hearing prostheses or otherwise a totally implantable medical device that is configured to execute one or more of the method actions detailed herein as noted above), and when (if) the CV readings result in a difference, such as a change in shape for example from one representing platinum to one more representative of iridium, that will be an indication that the electrode has worn to the depth where the iridium was deposited.

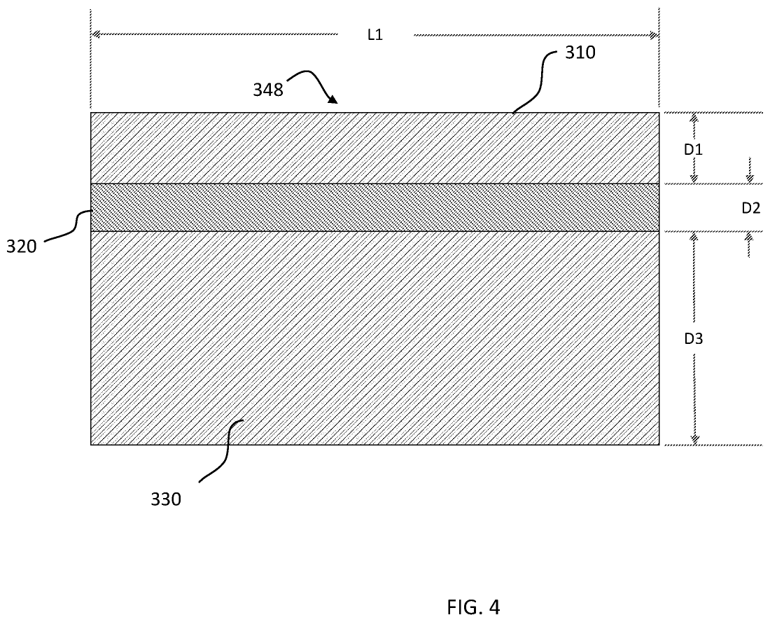

FIG. 4 presents a cross-sectional view taking through section 4-4 of FIG. 3 (and FIG. 2B, but without the carrier material shown), but without the backdrop shown. As seen, the relative thicknesses of the three sections are shown. Values of D1, D2 and D3 can be percentages of the summation of all those three values as will be understood. By way of example only and not by way of limitation, a value of D1 and/or D2 can be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 11, 12, 13, 14, 15, 16, 17, 18, 19, 20, 21, 22, 23, 24, or 25 percent, or any value or range of values therebetween in 0.01% increments of the summation of D1 plus D2 plus D3 (and/or any additional layers that may be present or any thicknesses that may be present), where D3 can be the remainder.

In an exemplary embodiment, D1 plus D2 plus D3 equals any value or range of values between 1 and 700 micrometers in 1 micrometer increment (e.g., 50, 22 to 71 micrometers, etc.). The value can be smaller or larger in some embodiments. For thin film, it can be 0.05, 0.1, 0.15, 0.2, 0.3, 0.4, 0.5, 0.6, 0.7, 0.8, 0.9, 1, 1.5, 2 or more or less μm, in 0.01 μm increments.

In an exemplary embodiment, L1 equals any value or range of values between 50 and 1000 micrometers in 1 micrometer increment (e.g., 200, 300 or 400 or 222 to 541 micrometers, etc.).

In an exemplary embodiment, the exposed area for current generation electrodes is any value or range of values from 10 to 500 μm² or any value or range of values therebetween in 1 micrometer increments. The area can be smaller or larger in some embodiments.

It is noted that in some embodiments, the above noted dimensions may be reduced by 5, 10, 15, 20, 25, 30, 35, 40, 45, 50, 55, 60, 65, 70, 75, 80, 85, 90, or 95% or more or any value or range of values therebetween in 1% increments, such as may be the case with cochlear implant electrode arrays that have 100 electrodes instead of 20 or 200 electrodes (the same current can be spread out over more electrodes, thus enabling smaller electrodes). For example, increasing the number of electrodes on a cochlear implant electrode array could result in a 10 fold reduction in area for a given electrode compared to some embodiments, which can be about 5, 6, 7, 8, 9, 10, 11, 12, 13, 14, 15 μm² or any value or range of values therebetween in 1 μm² increments. Moreover, if the electrodes are embedded into tissue, as opposed to offset, the electrodes could be smaller.

All the above said, spine stimulators and/or pacemakers can utilize larger electrodes such as, for example, electrodes that have surface areas of 0.1, 0.5, 0.75, 1, 1.5, 2, 2.5, 3, 3.5, 4, 5, 6, 7, 8, 9 or 10 mm² or or any value or range of values therebetween in 0.1 mm².

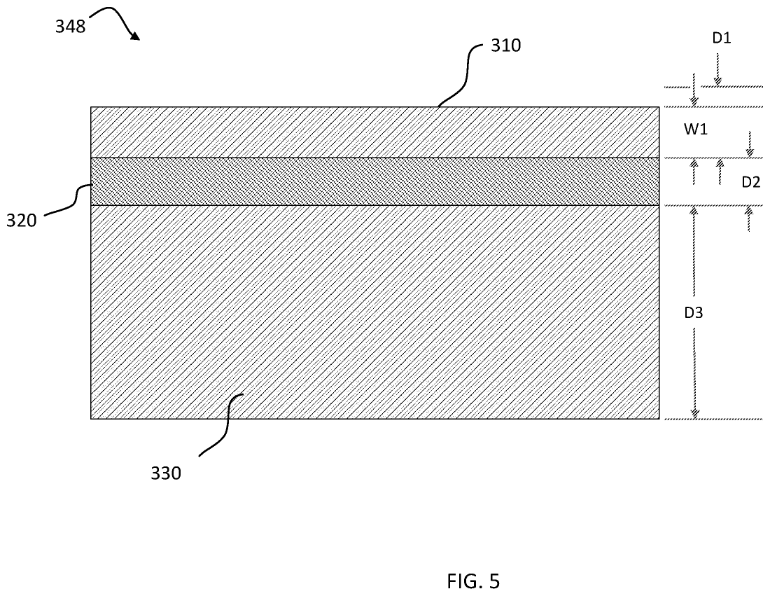

The above values are for a pristine electrode at the day of implantation. As the electrode is utilized or otherwise the longer that the electrode array is exposed to the ambient environment within the human being, such as for example perilymph within the cochlea, the outer layer of the electrode will wear over time. This is shown by way of example only and FIG. 5, which shows a thickness W1 (wear 1) that can result in an exemplary scenario 5 or 6 or 7 or 8 years or more after implantation (and use). The value of W1 may or may not be measured/measurable, but it is noted that in this exemplary embodiment, the electrical phenomenon associated with the teachings detailed herein would be the same or relatively about the same as that which would be the case for the pristine electrode shown in FIG. 4. For example, if W1 was equal to about 80% of D1, the electrode would operate the same or about the same for a given electrode charge, etc., (all other things being equal) as that which is the case for the pristine electrode (biofilms, etc., aside). This could also be the case of W1 was equal to about 20% of D1 for example or less even in some embodiments.

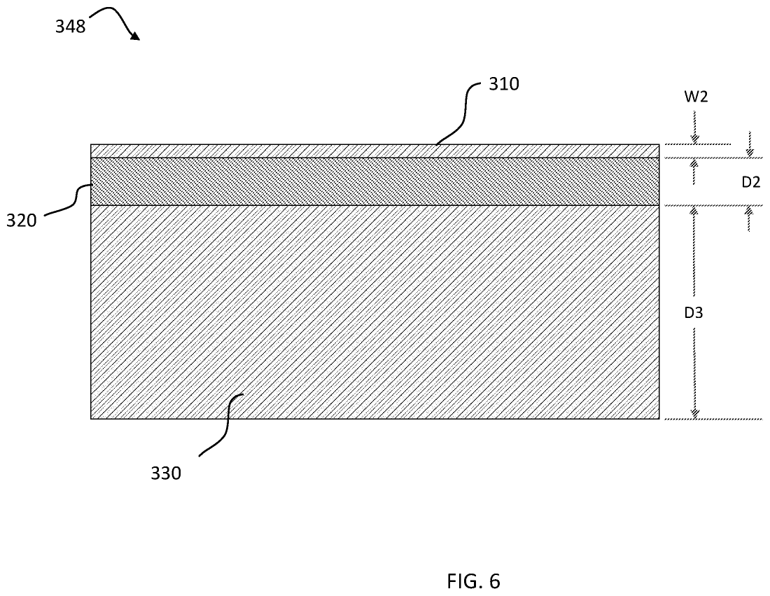

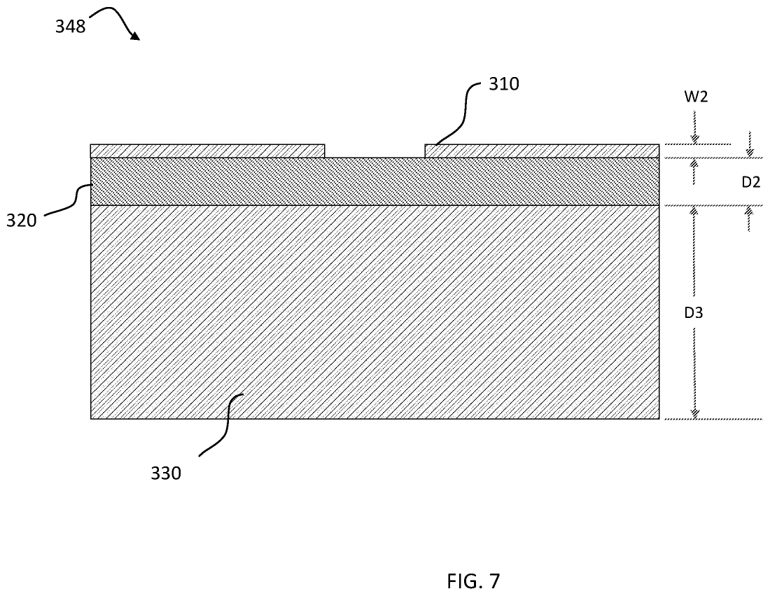

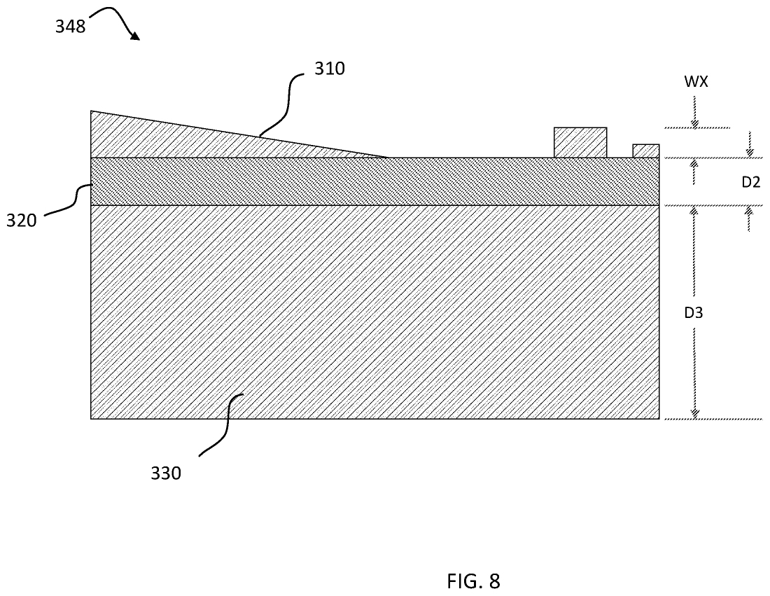

FIG. 6 depicts an exemplary scenario where the top layer 310 has a thickness of W2, where W2 is, say, 20% of D1. This could be at a temporal location years later from that of W1 noted above (5, 10, 20 years, for example), and could be a temporal location that is linearly extrapolatable temporal location where W1 existed. That said, owing to a change in body chemistry or owing to a change in the stimulation pattern owing to a change in the recipient's reaction to the cochlear implant (increased current needed, etc.), the resulting thickness W2 could exist of the temporal location much closer to that of W1, such as a year or two or three or less. Still, in at least some exemplary and scenarios, the performance or otherwise the electrical characteristics of the electrode array would still be about the same if not the same as that which is the case with respect to the pristine electrode of FIG. 4. Still, there will come a point where the thickness of layer 310 decreases to a point where the electrical phenomenon and/or the performance will change in a noticeable or otherwise measurable manner. This could exist when W2 gets to, say 1 or 2% of D2, or when a percentage of the surface area of the layer 320 becomes exposed to the ambient environment. FIG. 8 presents an exemplary embodiment where the passive dissolution and/or active dissolution of layer 310 is uneven. Here, it can be seen that there is a portion of layer 320 that is exposed to the ambient environment, with other portions that are still covered by the remnants of former layer 310. The remnants have variable heights which go by the height WX.

The percentage of the surface area of portion 320 that is exposed to the ambient environment could be variable or otherwise may be different for certain users and/or implementations or settings of the cochlear implant, at least with respect to resulting in a detectable or otherwise measurable phenomenon that can be harnessed to implement the teachings detailed herein. By way of example only and not by way of limitation, if at least 20 or 25 or 30% of the surface area of layer 320 becomes exposed to the ambient environment, this can result in a change of the performance features or the electrical phenomenon. Accordingly, in at least some exemplary embodiments, a discernible indication can exist where, for example, at least or equal to 5, 10, 15, 20, 25, 30, 35, 40, 45, 50, 55, 60, 65, 70, 75, 80, 85, 90, 95, or 100%, or any value or range of therebetween in 1% increments of the surface area 320 becomes exposed to the ambient environment, again, depending on the physiological nature of the human being and/or the stimulation or electrical current that is utilized and/or the sensitivity of the sensing devices are measuring devices within the body/that are part of the implant.

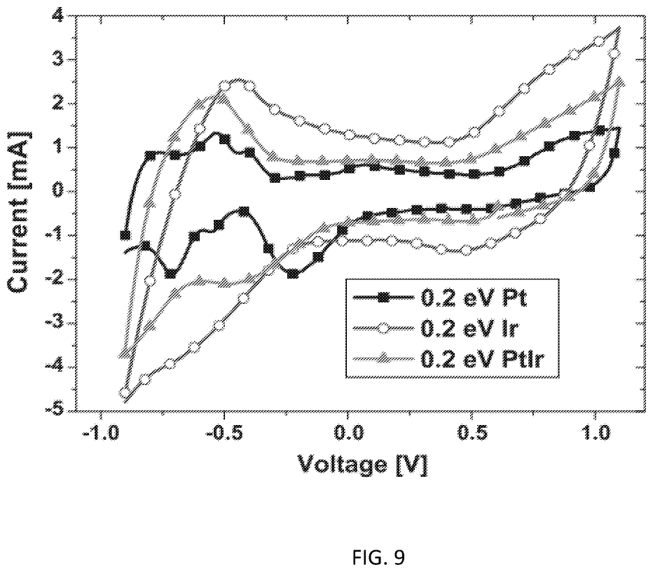

FIG. 9 presents an exemplary plot showing CV values resulting from sputtered platinum-iridium layers. This shows an exemplary cyclic voltammograms for platinum, iridium, and a platinum/iridium alloy. As can be seen, there are distinctive peaks for the respective materials, different for both materials, which occur at the same potential. In at least some exemplary embodiments, these peaks can be utilized to identify the ratio of each material present on the surface. That is, in an exemplary embodiment, by utilizing read electrodes of the electrode array or other electrodes, or otherwise by inferring or otherwise estimating the voltage levels or other electrical phenomenon, voltage versus current data can be obtained, and then analyzed, to determine the current wear status of the electrode. By way of example only and not by way of limitation, if after year/during year 1, 2, 3, 4, 5, and 6, the resulting plots show the curve for the platinum(s) (and the “testing”/data collection and/or analysis to arrive at those plots can be done every one, two, three, four, five, six, seven, eight, nine, 10, 11, 12 months or more, or fewer, or any value or range of values therebetween in one day increments), and then, after the seventh year (from implantation) or during such, say, at 7 years and 3 months after implantation) the resulting plot shows the curve for iridium (or iridium/platinum, where layer 320 is a platinum alloy or an iridium alloy), it can be inferred that the overlaying layer 310 has worn off or otherwise a significant amount of that layer has a worn off. In an exemplary embodiment, this can be used to deduce the estimated life expectancy of the cochlear implant electrodes with respect to passive dissolution and/or active dissolution, or otherwise wear. By way of example only and not by way of limitation, if layer 310 made up 5% of the total thickness of the electrode, it can be deduced that the electrode will ultimately wear out in at least 114 years, assuming a linear wear rate. That said, taking into account the fact that the surface area will be reduced with wear and/or that there comes a point that even if there is some material of the underlying electrode remaining, the electrode becomes ineffective or otherwise the minimal thickness of the remaining electrode material will result in a quickening of the passive dissolution rate and/or active dissolution rate, etc. and/or taking into account conservative principles utilizing safety margins, the expected lifetime of the electrode could be 80 years hence (using a factor of safety of 0.7 for example—factors of safety can be 0.1, 0.2, 0.3, 0.4, 0.5, 0.6, 0.7, 08, 0.9 or 1.0 or any value or range of is therebetween in 0.1 increments). In an exemplary embodiment, the minimum thickness of, for example, 5, 6, 7, 8, 9, 10, 11, 12, 13, 14, 15, 16, 17, 18, 19, or 20% of D3, or any value or range of values therebetween in 0.1% increments could be determined to be the limit of wear (akin to an automobile brake pad evaluation—the brake pad can still be used after an inspection, but there is a minimum thickness where it is determined that the break patch no longer be utilized). Accordingly, the expected lifetime of the electrode could be then reduced to 60 years from that date, for example.

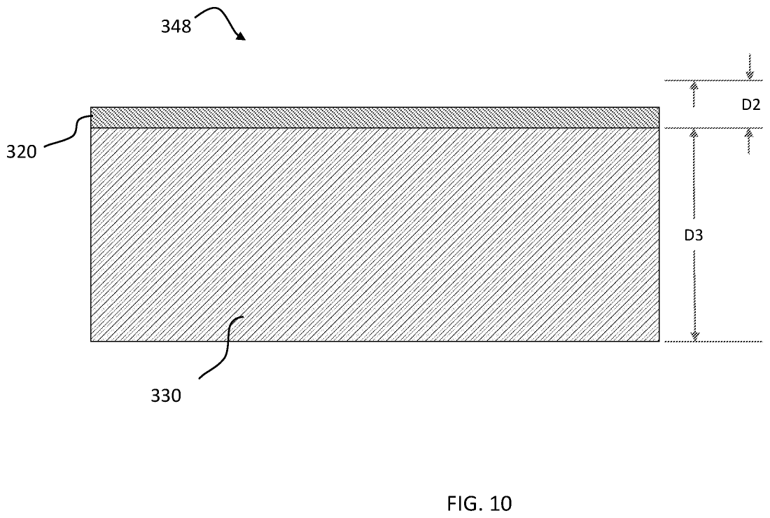

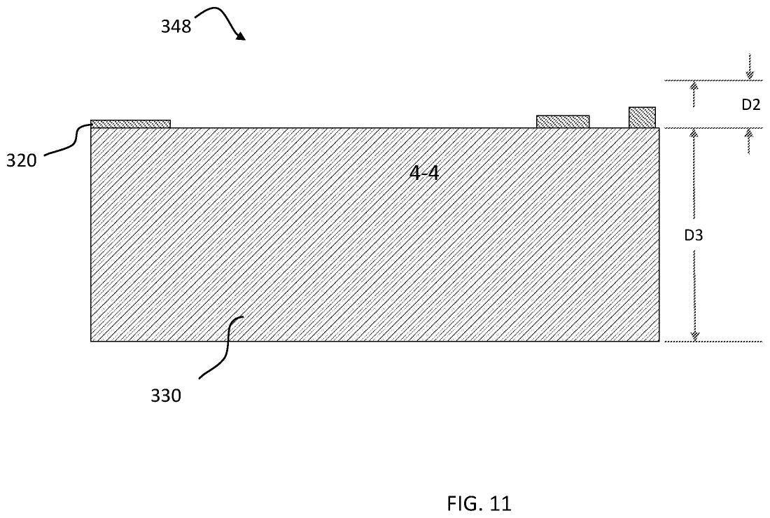

Note also that in an exemplary embodiment, further testing and/or evaluation or otherwise monitoring can be executed to identify the change in the plot when the layer 320 wears to the point where the curves change again owing to the underlying platinum layer 330. Briefly, FIG. 10 depicts a wear scenario for the iridium layer 320 where the iridium layer has worn out over half the original distance D2. Then, at some time later, at least a portion of the underlying platinum layer 330 becomes exposed, as shown in FIG. 11, which then changes the curves back to a platinum curve (or towards a platinum curve). This can be utilized to fit the curve or the like to further estimate the expected wearout date of the electrode.

As can be understood by the plot of FIG. 8, layer 320 can be a platinum alloy or a platinum-iridium alloy or an iridium-platinum alloy. Also, in an exemplary embodiment, layer 310 and/or 330 can be a platinum alloy and/or a platinum-iridium alloy or an iridium-platinum alloy. Note also that in an exemplary embodiment, one or more of the layers can be an iridium layer. Also, other materials can be utilized. Further, the concentrations of materials can be utilized, and materials can be doped with other substances that still enable the electrode to work or otherwise operate, but also result in a change on electrical characteristics or some other phenomenon were otherwise the creation of a phenomenon that can be measured or otherwise detected to be used in the methods disclosed herein to determine the wear state of an electrode or otherwise estimate the ultimate wearout date of the electrode.

Briefly, with respect to methods of manufacture, in an exemplary embodiment, iridium is deposited at the desired depth through additive manufacturing on to the underlying platinum layer 330, either as a pure metal or as an alloy, or as a coating (for example, sputter coated), electrodeposited or through any of the surface deposition methods that can have utilitarian value, the iridium has been, in this embodiment, added at a particular depth, followed by further platinum deposition (layer 310). Further, the iridium layer can be added as a deposit on the surface of an existing electrode (existing design electrode). It could be manufactured by rolling thin films of Pt, Ir and Pt together and then forming an electrode from the resultant foil (e.g., by bending/forming a band or half band from a flat plate of the layered material.

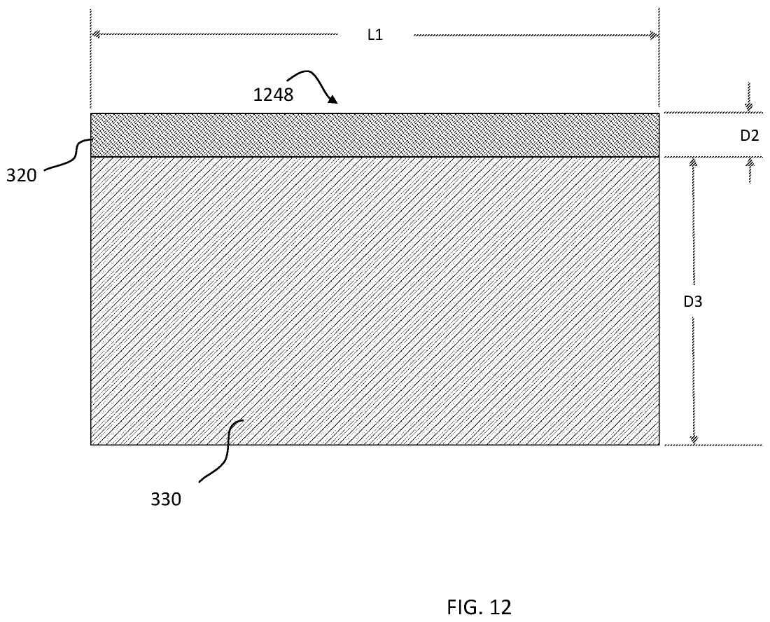

FIG. 12 presents an alternate exemplary embodiment where the electrode 1248 is only a two layer electrode. Here, there is no layer 310. There is only layer 320, which is a platinum or platinum-iridium or iridium platinum layer etc., and then layer 330, which is a layer that is distinct from layer 320, which can correspond to layer 330 above. Note that layer 320 can correspond to layer 320 above. The thicknesses of layers can be different with respect to that detailed above, at least with respect to layer 330, owing to the fact that there is no layer 310. The thicknesses of layers can be the same with respect to that detailed above. Further, where the thickness of layer 320 is different than that which was the case above, the thickness of layer 320 can be different than that which is the case with respect to layer 310 above and/or with respect to layer 320 above. The thickness can be greater or smaller than that which was the case detailed above. In an exemplary embodiment, such as, for example, where the layer 320 has inherent wear resistance than that which would be the case if there was a layer 310 (e.g., iridium, in at least some exemplary scenarios, can resist active dissolution and/or passive dissolution, in at least some exemplary embodiments or otherwise scenarios of use, at a markedly superior manner than platinum—this can all be taken into account with respect to the design of the electrode and/or with respect to the evaluations/measurements—note that this is also the case with respect to the embodiments that utilize the three layered electrode—the iridium layer may take longer to dissolve and/or erode, and this would be known or otherwise can be factored into the overall curve fitting—the fact that the wear rate of the iridium layer will be different than the wear rate of the underlying platinum layer can be taken into account to estimate or otherwise forecast the ultimate wearout date of the electrode (a weighting may be applied to the length of time that it takes to wear through the iridium layer).

In at least some exemplary embodiments, the electrode has two, three, four, five, six, seven, eight, nine, and/or 10 or more distinct layers, at least one, two, three, four, five, six, seven, eight, nine, and/or 10 layers are different with respect to material properties from at least one or both adjacent layers.

In view of the above, it can be seen that in at least some exemplary embodiments of the electrode array, the at least one electrode has a first layer of material and a second layer of material different from the first layer of material, the second layer of material being below the first layer of material relative to an ambient environment of the electrode array, the different layers enabling the in vivo analysis.

Conversely and/or in addition to this, in an exemplary embodiment, the at least one electrode has a first layer and a second layer having a different structure than the first layer, the second layer of material being below the first layer of material relative to an ambient environment of the electrode array, the different structure enabling the in vivo analysis. This difference in structure is differentiated from a difference in material, at least as that phrase “structure” is used herein.

In an exemplary embodiment, the implantable electrode array has at least one electrode, and the at least one electrode has a first layer and a second layer, the first layer has a porosity different from the second layer, the second layer is below the first layer relative to an ambient environment of the electrode array, and the different porosity enables the in vivo analysis.

In an exemplary embodiment, additive manufacturing is used to build a thick film (e.g. 50 μm deep) electrode contact which has varying degrees of porosity at different depths. In this regard, the above-noted layers may be more generally considered more generalities as, for example, the difference between the troposphere and the stratosphere has a definition but the transition is not a distinct transition—in other embodiments, the layers have distinct transitions from each other. Embodiments can include monitoring passive dissolution and/or active dissolution of a layer of material, which layer of material is at a known depth within the electrode (as is the case with the layers of different material noted above). As with the embodiments utilizing different materials, this layer can be located relatively near the surface of the electrode, as this can have utilitarian value with respect to indicating the rate of passive dissolution and/or active dissolution, or otherwise wear, earlier rather than later in the implant's life. As the electrode dissolves/erodes, etc., it does so from the surface downward, exposing lower layers of the electrode as time goes by. When the region of different porosity is exposed, the electrochemical behavior of the surface changes. In the case where the layer 320 has a greater porosity than layer 310, greater porosity exposes a different surface area of the electrode to the ambient environment. This would expose a greater surface area relative to that which was the case relating to layer 310, at least about immediately before the layer 310 wore down to expose layer 320 (because of the cylindrical nature of at least some embodiments of the electrodes, the surface area will be reduced irrespective of the change in ferocity, because the radius will be reduced). Conversely, in the case were layer 320 has a lower porosity than layer 310, the wear of layer 310 would also exposes a different surface area of the electrode to the ambient environment (a lesser surface area relative to that which was the case relating to layer 310, or at least a distinct change in the surface area relative to that which was the case immediately before the layer 310 wore down to expose layer 320 (this is also the case where the layer 320 has the greater porosity, albeit in reverse—the point is, that the distinct change in the surface area can be utilized as an indicator).

In at least some exemplary embodiments, the porosity of the majority of the electrode would be established for maximum utility vis-à-vis the combination of electrochemical performance (in at least some exemplary embodiments, the bulk of the electrode has a relatively higher porosity because, again in at least some exemplary embodiments, such increases surface area from which the electrical current can flow into the ambient environment, which reduces electrode impedance and “polarization” in at least some exemplary embodiments, both parameters that are minimized to the greatest extent possible). Thus, in an exemplary embodiment, the region of lower porosity is exposed for a relatively limited thickness of the electrode's overall depth. The amount could be not enough to weaken/degrade the performance of the electrode in a significant or noticeable manner, but enough to register when this section of the electrode is exposed due to passive dissolution and/or active dissolution. When this lower porosity region is exposed, in at least some exemplary embodiments, this lower porosity region causes a decrease in the exposed surface area of the electrode.

Electrochemical measurements can be used to detect the exposure of the decrease in area and thus determine that the region is exposed. (And thus that the overlying layer has worn away.) Knowing the stimulation history of that electrode (which can be the case of the device records/logs such features, which in some embodiments the medical devices due, and permit the data to be downloaded or transferred for analysis, and/or the medical device itself can evaluate the stimulation history of that electrode), embodiments include deducing/predicting/estimating the expected remaining lifetime of the electrode (or other electrodes, by extrapolation/proxy).

One electrochemical method that can be utilized in at least some exemplary embodiments that can enable the determination of the “real” surface area of an electrode (as opposed to its geometric area—again, the porosity is what drives the real surface area, and the change in porosity is what provides the indicator that can be utilized to determine the wear status and/or the wear rate, etc.) is the Charge Storage Capacity or CSC. The CSC can be utilized in at least some exemplary embodiments to provide a measure of the reversible electrochemical reactions that occur at the surface of an electrode. In some embodiments, this is utilized to provide a measure of the porosity of the material at that time in the electrode's life. In at least some exemplary embodiments, the CSC is generally proportional to the real surface area of an electrode and can therefore be used as a measure of the real surface area. In at least some exemplary embodiments, it can be calculated as the area with a curve traced using a method called a cyclic voltammogram.

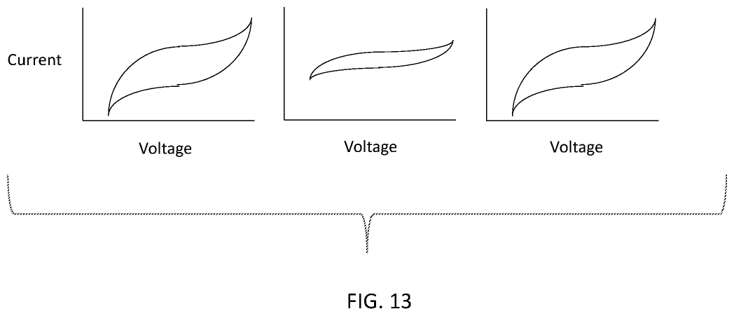

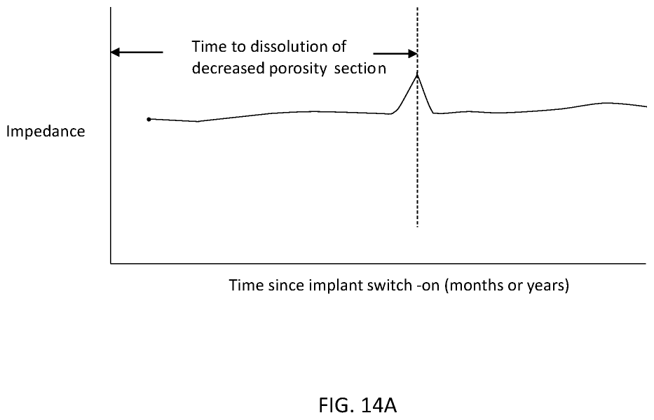

FIG. 13 presents exemplary cyclic voltammograms performed at different times during the electrode's life. The middle plot depicts the phenomenon that is seen when the layer 320 of higher porosity material relative to layer 310 and 330 is exposed. The left and right plots depict the phenomenon that is seen when layer 310 is present, and then when layer 330 is exposed to ambient environment because of the active dissolution and/or passive dissolution of the overlying layer 320. Because the implant is configured to be able to obtain the voltage and/or current and/or impedance data related to the electrodes or areas proximate the electrodes, the data can be presented or otherwise arranged according to the plot seen in FIG. 13, and then, when evaluated, or otherwise compared to each other, a determination about the wear status and/or wear rate of the electrode can be made.

An alternative embodiment that can be utilized to register the porosity of the electrode (at least the portion that is immediately exposed to the ambient environment) is to measure the impedance of the electrode at various times during the electrode's life, such as any of those detailed herein. When an area of lower porosity is uncovered, the impedance will increase (and the opposite occurs when an area of higher porosity is uncovered). The impedance can be straightforward to measure in a cochlear implant, or other medical device, such as by utilizing the techniques of the aforementioned publications by Carter, Pawsey and Melman, and/or variations thereof.

Impedance measurements may not, in at least some instances, enable the provision of as accurate a measure of porosity as the CSC. In this regard, CSC takes into account the impedance measures features of the tissue in the vicinity of the electrode surface as well as the surface itself. Nevertheless, because the impedance of a statistically significant number of recipients is relatively stable over time, this method can provide a utilitarian method for determining the electrode life.

It is noted that in some embodiments where the layer portion of the electrode that becomes exposed owing to wear of the overlying layer or portion of the electrode has a higher porosity, relative to the overlying layer, the increase in porosity of the electrode exposed to the ambient environment will result in lower impedance but high CSC, and visa-versa.

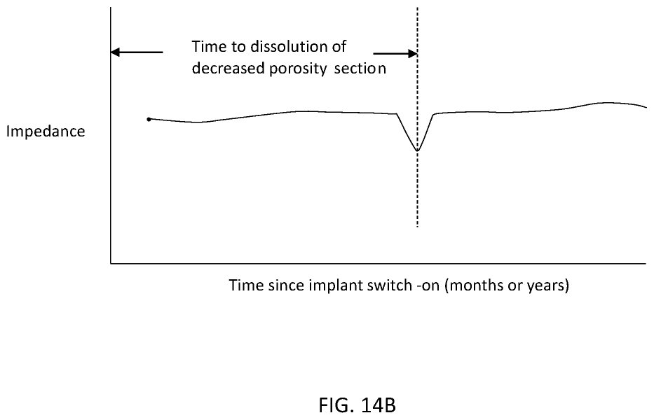

FIG. 14A presents a conceptual graph of how impedance increases when dissolution exposes a region of increased porosity compared with the porosity of the surrounding rest of the electrode contact. FIG. 14B presents the opposite.

In an exemplary embodiment, the electrode array has sections that have different porosities. For example, the at least one electrode has a first layer and a second layer, the first layer is made of different material and/or has a porosity different from the second layer, the second layer is below the first layer relative to an ambient environment of the electrode array and the different material and/or different porosity enabling the in vivo analysis.

In at least some exemplary embodiments, the electrode has two, three, four, five, six, seven, eight, nine and/or 10 or more distinct layers, at least one, two, three, four, five, six, seven, eight, nine and/or 10 layers are different with respect to porosity from at least one or both adjacent layers.

Embodiments can include electrodes that have a roughened surface, and thus the roughened surface can result in a phenomenon that corresponds to variable porosity electrodes. Therefore, the techniques detailed herein can be used to monitor the expected life of a roughened electrode. At the start of the electrode's life, the electrode has a high roughness (which thus can give the electrical appearance of a high porosity portion), and the impedance is low (relative to that which would be the case in the absence of the roughness), and its CSC is high (again, relative). In some embodiments, as the roughness starts to dissolve/erode, or otherwise wears away, and the electrode becomes lower in roughness, and adopts the general overall porosity, the electrode's impedance will increase relative to the virgin rough surface and its CSC will decrease (again, relative to the virgin rough surface). The rate at which this occurs can be used to measure the rate of expected dissolution of the electrode and therefore its expected life.

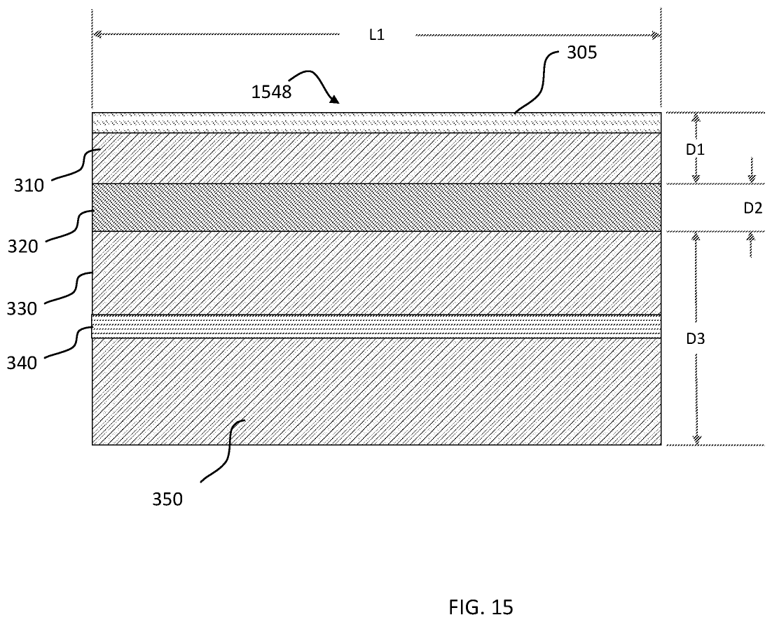

Combinations of the above can be used in a distinct manner. FIG. 15 presents an exemplary cross-section of an electrode 1548. As shown, there is a portion 305 that constitutes a roughened surface area. This portion can have a thickness that is greater than less than or equal to 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50% or more, or any value or range of therebetween in 0.1% increments of the overall value of D1. Layer 310 corresponds to the layer 310 detailed above, which can have the “standard” material properties and/or structure of the majority of the electrode or the plurality of the electrode (relative to thickness, and/or volume and/or mass). In an exemplary embodiment, the plurality and/or majority of the structure and/or material of the array makes up 55, 60, 65, 70, 75, 80, 85, 90, 91, 92, 93, 94, 95, 96, 97, 98, or 99% or more or any value or range of values therebetween in 0.1% increments of the mass and/or thickness and/or volume for a given measurement regime. Thus, in an exemplary embodiment, 83.5% of the mass of the electrode array is made up of distinct platinum sections having a porosity of a given value that has a surface that is not roughened. Section 320 can correspond to a material and/or structure that is different from that of section 310. In this exemplary embodiment, the material is different. Layer 330/section 330 has the same structure and material as layer/section 310, and is included in the aforementioned plurality and/or majority, and this is also the case with respect to layer/section 350. Section 340 can have a material and/or structure that is different from that of section 330. In this exemplary embodiment, the porosity can be different.

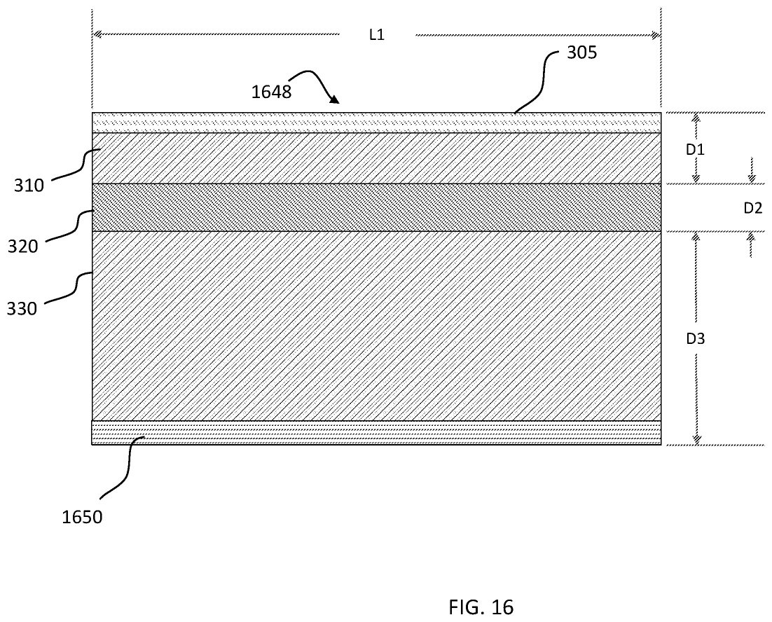

FIG. 16 presents another exemplary embodiment of an electrode 1648. Here, the bottom most/inner most section 1650 is made out of iridium or an iridium alloy and/or a platinum-iridium alloy or any other material that is different than that of layer 330, which can be a platinum or platinum alloy and/or has a porosity that is markedly different from that of layer 330 (different sufficiently to result in the above-noted phenomenon when the layer is exposed to the ambient environment). In this exemplary embodiment, this can provide a final warning or otherwise an indication that the “end is near.” Also, the makeup of section 1650 can be something that resists active dissolution and/or passive dissolution in a manner greater than that of the other layers, thus, “buying time” for various remedial actions to be taken, such as those detailed herein.

In an exemplary embodiment, section 1650 and/or section 340 has a thickness that is greater than less than or equal to 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50% or more, or any value or range of therebetween in 0.1% increments of the overall value of D3.

Some embodiments include a method for separating the electrode surface and tissue/ambient environment contributions towards the impedance associated with the electrode through the use of Electrical Impedance Spectroscopy (EIS). EIS (as noted above in the earlier Carter patent application publication) applies voltages and measures currents (or vice versa) over a range of frequencies. Low frequencies can be utilized, and, in some embodiments, the impedance of the electrode interface becomes dominant at low frequencies. In at least some exemplary embodiments, low frequency techniques such as cyclic voltammetry (from which the CSC is derived, running at, for example, 50-150 mV per second) can be utilized for extracting information about the electrode surface. In some embodiments, a full set of EIS measurements is obtained utilizing the medical device. In some embodiments, a simpler form of “pseudo EIS” is performed by taking impedance measurements at different pulse widths. In particular, long duration (e.g., up to 1 ms) pulses will give a representation of the real surface area or porosity of the interface.

Thus, in an exemplary embodiment, the at least one electrode is configured to trigger a measurable change in a charge storage capacity of the electrode upon a passive dissolution and/or active dissolution amount of the at least one electrode, the measurable change enabling the in vivo analysis. In an exemplary embodiment, the change is a change that is at least and/or equal to 5, 6, 7, 8, 9, 10, 11, 12, 13, 14, 15, 16, 17, 18, 19, 20, 21, 22, 23, 24, 25, 30, 35, 40, 45, 50, 55, 60, 65, or 70% or more, or any value or range of values therebetween in 1% increments from the measured amount prior to the change. In an exemplary embodiment, this could be, for example, the area within the enclosed curves of the charge storage capacity. In an exemplary embodiment, this could be, for example, the peaks established by the curved and/or the wet slow curves and/or the heights of the curves and/or the mean, median and/or mode of the geometric center of the curves (the enclosure).

In an exemplary embodiment, the at least one electrode is configured to trigger a measurable change in a cyclic voltammetry spectrum of the electrode upon a passive dissolution and/or active dissolution amount of the at least one electrode, the measurable change enabling the in vivo analysis. In an exemplary embodiment, the change is a change that is at least and/or equal to 5, 6, 7, 8, 9, 10, 11, 12, 13, 14, 15, 16, 17, 18, 19, 20, 21, 22, 23, 24, 25, 30, 35, 40, 45, 50, 55, 60, 65, or 70% or more, or any value or range of values therebetween in 1% increments from the measured amount prior to the change. In an exemplary embodiment, this could be, for example, the peaks established by the curved and/or the wet slow curves and/or the heights of the curves and/or the mean, median and/or mode of the values of the curves.

By using the electrodes herein, for example, such provides methods that provide a patient-specific way to minimise the risk of premature device degradation and replacement, and does not relay on estimates of population average wear out rates.

In view of the above, it can be seen that exemplary embodiments include recipient specific methods that minimize and otherwise manage electrode degradation, such as minimizing the risk of premature device degradation and/or the requirement to replace the device.

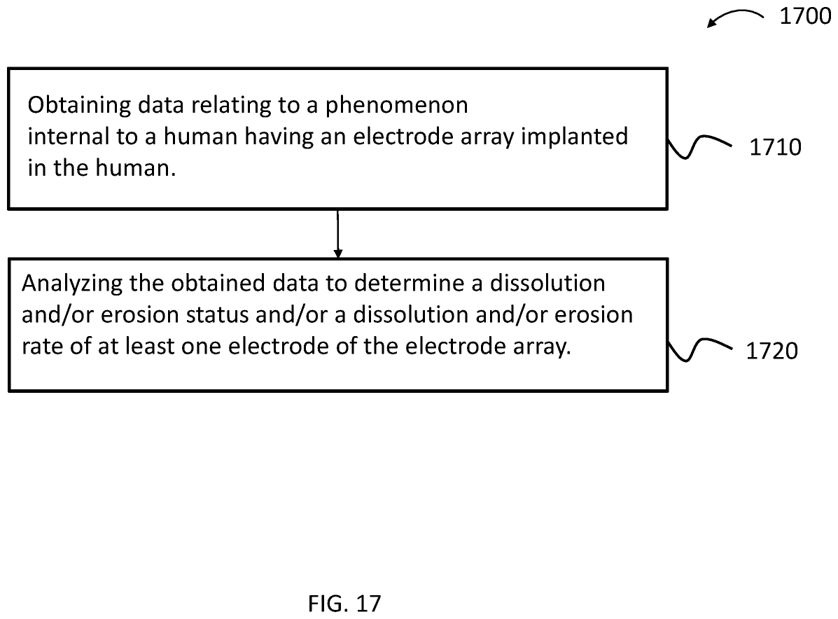

FIG. 17 presents an exemplary algorithm for an exemplary method, method 1700, according to an exemplary embodiment. Method 1700 includes method action 1710, which includes the action of obtaining data relating to a phenomenon internal to a human having an electrode array implanted in a human. In an exemplary embodiment, the electrode array is a cochlear implant electrode array implanted in a cochlea. Method action 1700 further includes method action 1720, which includes analyzing the obtained data to determine a passive dissolution and/or active dissolution status and/or a passive dissolution and/or active dissolution rate of at least one electrode of the electrode array. In this exemplary embodiment, the action of obtaining data is executed, at the time of obtaining data, non-invasively. In this regard, such as where the obtained data is obtained utilizing the implanted portion of the cochlear implant, even though an invasive procedure was required to implant the implantable portion, because the method is qualified at the time of the action of obtaining data, if this occurs after implantation/after the implantation procedure is completely completed, the data is obtained non-invasively. In an exemplary embodiment, the action of obtaining data is executed, again at the time of obtaining data, minimally invasively. In this regard, as opposed to the action of implanting the implantable portion of the cochlear implant of FIG. 1, which would be an invasive procedure, a needle to access, for example, a blood sample, such as accessed from a vein in one's arm, would be minimally invasive.

Briefly, with respect to a status, this can correspond to the percentage of the electrode that has dissolved and/or eroded relative to the virgin electrode/electrode at implantation. This can correspond to a mass and/or volume and/or thickness percentage. In fact, with respect to status, in some embodiments, this can correspond to an actual dimensional amount, such as the number of milligrams or micrograms of the electrode that are present and/or the thickness of the electrode, etc. Because there is an understanding of the virgin/original dimensions/properties of the electrode, the status can be determined. That said, in some other embodiments, the status can be determined based on an estimate or otherwise a sufficient educated guess as to what the original/virgin dimensions/properties of the electrode were. In an exemplary embodiment, this can be based on industry standards and/or expertise, etc. By rough analogy, for a given mission profile and/or functionality of a fighter plane, the F-15 and the MiG-29 wound up looking about the same and having relatively similar properties. In the same vein, a person of skill can extrapolate the properties of the electrode at implantation based on the desired functionality of that electrode at implantation, etc.

In an exemplary embodiment, any one or more of the actions detailed herein can be executed upon a triggering event associated with the wear status and/or wear rate of the electrodes. In an exemplary embodiment, any one or more of the actions detailed herein are triggered upon a determination that the wear status of the electrode is greater than, less than and/or equal to 99, 98, 97, 96, 95, 90, 85, 80, 75, 70, 60, 50, 40, 30, 20, 150, 14, 13, 12, 11, 10, 9, 8, 7, 6, or 5 percent, or any value or range of values therebetween in 1% increments of the total value of the virgin electrode (e.g., mass, volume, etc.). In an exemplary embodiment, any one or more of the actions detailed herein are triggered upon a determination that the wear rate of the electrode is greater than and/or equal to 30, 20, 15, 10, 9, 8, 7, 6, 5, 4, 3, 2, or 1% or any value or range of values therebetween in 1% increments per year and/or per decade.

In an exemplary embodiment, the rate of passive dissolution and/or active dissolution can be an extrapolated or estimated value based on known data that can have utilitarian value in a statistically sufficient manner to forecast the future. By way of example only and not by way of limitation, for a given current and/or pulse regime, if after seven years, the electrode has experienced a 10% passive dissolution and/or active dissolution (meaning that 90% of the beginning amounts of the electrode remains, by thickness and/or by mass and/or by volume), the passive dissolution rate and/or active dissolution rate would be 1/10^{th} of original unit per 7 years). That said, in some embodiments, one can take into account, for example, an initial break in period such as where the roughened surface has a slightly different passive dissolution and/or active dissolution rate, has a faster or slower dissolution rate. Thus, the passive dissolution and/or active dissolution rate could be 1/9^{th} or 1/15^{th} per 6 years, for example (discounting the first year as a break-in period).

In an exemplary embodiment, the obtained data can correspond to the aforementioned voltage and/or impedance and/or current readings. In an exemplary embodiment, the obtained data can correspond to the cyclic voltammetry spectrum data and/or the charge storage capacity data noted above.

In an exemplary embodiment, method action 1720 can be executed in an automated and/or a manual fashion. As noted above, the neural networks and/or a trained expert system can be utilized to automatically analyze the obtained data. Moreover, in some embodiments, the action of analyzing is executed automatically by a prosthesis of which the electrode array is a part (e.g., by the implantable portion of the cochlear implant and/or by the external component of the cochlear implant, etc.). Accordingly, there are computing devices and/or medical devices along these lines that are configured to execute method action 1720.

In an exemplary embodiment, the obtained data is obtained using impedance based techniques. In an exemplary embodiment, the phenomenon is based on current and/or voltage measurements associated with at least one electrode of the cochlear implant electrode array. In an exemplary embodiment, the obtained data is obtained utilizing impedance spectroscopy.

In an exemplary embodiment, baseline data is collected, either initially shortly after implantation or within a reasonable time after implantation (so as to, for example, allow scar tissue and/or fibrous tissue to grow, which growth tends to occur relatively shortly after implantation with respect to the overall life of the implant). In an exemplary embodiment, the aforementioned baseline data is collected at the time of implantation or during the same day of implantation, or within 1, 2, 3, 4, 5, 6, 7, 8, 9, 10, 11, 12, 13, or 14 days from implantation and/or within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks, or months, or any value or range of values therebetween in one day increments. This baseline data can correspond to “standard” impedance related data for example. A baseline database of faradaic related values can be obtained during this time period for example. Then, during the aforementioned data collection/testing periods that can occur after the baseline data is established (and/or can be utilized to further increase the baseline data, if there is no change for example), the data obtained can be compared to the baseline data, and if there is a change in a variable from the stabilized baseline data, such can be indicative of a change in the wear rate and/or the wear status of the electrode contact.

In an exemplary embodiment, faradaic components can be isolated during the baseline data development and during the testing periods, and these components can be compared or otherwise evaluated to determine whether or not respective components are effectively different from each other and thus indicate that the electrode has experienced wear since the baseline data was developed.

In an exemplary embodiment, potential changes in polarization can be indicative of wear of an electrode. Accordingly, embodiments can include developing a baseline data set of polarization values associated with the electrodes in general, and how those are utilized in particular, and if a change in polarization is detected, such can be an indication that the electrode has experienced wear.

Embodiments above are focused primarily on how the electrodes can be utilized to create or otherwise establish electrical phenomenon that can be utilized to determine the rate and/or status of wear of an electrode. Embodiments also can include the utilization of statistical methods and/or other techniques. By way of example only and not by way of limitation, in an exemplary embodiment, the phenomenon of method action 1710 is body chemistry. As noted below, in an exemplary embodiment, the data that is obtained in method action 1710 is obtained noninvasively and/or minimally invasively. With respect to the former, in an exemplary embodiment, the implanted medical device can include sensors or the like that can detect body chemistry, either directly or by latent variable relationships (e.g., an impedance and/or voltage reading and/or current reading for a given charge may be indicative of a chemical makeup of perilymph within the cochlea and/or substance within the cochlea. Also as noted above, the data can be obtained in a minimally invasive manner. In this regard, in an exemplary embodiment, a blood sample or the like can be taken utilizing a syringe. Oxygen sensors can be utilized, which can be utilized via a noninvasive and/or minimally invasive device (monitors that attached to a finger, etc.). Moreover, in some embodiments, patch like devices, such as those utilized to monitor blood sugar levels etc., can be utilized.

In an exemplary embodiment, biopsies of blood or CSF or perilymph are used as a latent variable that provides an indicia of the rate and/or a state of wear of the electrode contact. Measurements can be made therein associated with biopsies of blood, etc., at regular and/or periodic or on periodic and on regular intervals, and the analysis or otherwise evaluations of these measurements can be utilized to provide latent variables indicative of the state of the electrodes.

Note also that in at least some exemplary embodiments, a recipient's medical history can be utilized to further ascertain or otherwise estimate a state of wear of the electrodes. In this regard, in an exemplary embodiment, methods include evaluating medical records of a recipient regarding his or her condition and/or past conditions and/or treatments and/or past treatments. Any one or more of these can be utilized in some embodiments to estimate or otherwise ascertain the wear rate and/or status of the electrode.

Note that in at least some exemplary embodiments, a trained neural network or an expert system can be utilized. In some embodiments, data from various recipients can be obtained and/or is otherwise available, and artificial intelligence or the like can be utilized to ascertain a pattern or a relationship between the various features detailed herein that can be utilized to provide an indicia of the wear rate and/or wear state of an electrode. Consistent with techniques that utilize artificial intelligence, the computing system and/or algorithms or other arrangements that can be utilized may not necessarily be evaluated to determine a strict cause-and-effect relationship between electrode wear states and/or rates and the variables inputted into the system. This is commonly accepted at this point in the year 2021. The point is that any computing system that can be provided with data or otherwise provided with variables relating to a person that has an implant with an electrode that will provide an indicia of the electrode wear rate and/or status can be utilized to execute at least some of the method actions herein, even if the system cannot be analyzed to determine exactly how that expert system or otherwise the train system, or otherwise the computer system or algorithm is arriving at its conclusions.

In at least some exemplary embodiments, the obtained data is an image of an electrode of the electrode array obtained while the electrode array is implanted in the human (e.g., in a cochlea). By way of example only and not by way of limitation, the amount of remaining electrode material, such as platinum for example, can be monitored using plain film x-rays. and/or CT scans. Some exemplary embodiments include implanting electrode contacts that are configured to show up in x-ray devices and/or CT scans that are commonly available in the United States of America on Mar. 20, 2021. By way of example only and not by way of limitation, electrode rings can be designed and manufactured that, when implanted, will show up show up sufficiently clearly in x-rays and CT scans. In some embodiments, images using the aforementioned technologies and/or variations thereof or other technology that can be utilized to noninvasively obtain images of the electrodes are obtained and those images can be analyzed, including utilizing computer analysis that can electronically evaluate the amount of material of a given electrode that is present (or absent). In some embodiments, the virgin/pristine or otherwise the original design of the electrode is known, and thus the data from the resulting image can be compared to the baseline (new) electrode to determine the current wear state and/or wear status of the electrode. In an exemplary embodiment, the intensity of the image can vary based on the amount of electrode material that is present/that is no longer present. The intensity of the images can be utilized to gauge the wear rate and/or wear status. In an exemplary embodiment, the images taken over a period of time, such as any of the temporal testing. Detailed herein, can be compared, and a reduction in the intensity can be indicative of an electrode that is wearing. The reduction in intensity over time can be plotted to determine the ultimate life expectancy of the electrode. Alternatively, and/or in addition to this, the raw intensity can be compared to data indicative of what the intensity should be for a pristine electrode and/or a non-pristine electrode that has been subject to wear, but which wear is de minimis or otherwise within design parameters.

In an exemplary embodiment, the composite electrode concept can be utilized in combination with the offer mentioned imaging technologies. In an exemplary embodiment, the porosity features and/or the composite nature of the electrode will show up on the imaging devices. Alternatively, and/or in addition to this, a composite electrode can include material that will provide a different indication, or more accurately, a discernible indication, on the imaging techniques utilized herein. For example, an electrode could include a film or layer of a radioactive material that is safe to the recipient, but will show up on the imaging, at least if such layer is exposed.

In at least some exemplary embodiments, the images that are taken are taken judiciously. By way of example only and not by way of limitation, at least some believe that they are can be utilitarian value with respect to limiting the exposure of a human being to x-rays. Accordingly, the image is would be taken, in some embodiments, only after a determination has been made that the electrodes are candidates for early passive dissolution or active dissolution relative to a statistical base line and/or after a certain lifetime that is statistically based where the likelihood of an electrode having experienced a significant amount of wear is greater than that which would otherwise be the case. With respect to the former, such can be the case with a user of a cochlear implant that utilizes high charge values relative to the average charge values across a given population. The higher charge values being more likely to result in earlier wearout of the electrode relative to that which would be the case for lower charge values. With respect to the latter scenario, such can be the case with a user of a cochlear implant who has had the cochlear implant for a statistically high number of years, such as 20 or 30 or 40 years. That is, the electrode has been in the body of the human being for a while, and thus has been used for a while, and thus like anything that is utilized for lengthy temporal period relative to something that is newer, it will experience wear. Accordingly, in an exemplary embodiment, the actions of utilizing imaging would be executed after a determination that other latent variables or otherwise other data indicates that the electrodes will experience wear or otherwise may be in a state of passive dissolution and/or active dissolution that could impair the functionality thereof in the short order or otherwise has already impair the functionality thereof.

In an exemplary embodiment, the imaging techniques detailed herein can be utilized as a “final” test to determine whether or not remedial action is utilitarian. For example, if other data indicates that the electrodes have experienced significant dissolution (e.g., the electrical phenomenon measurements indicate a reduced surface area, etc.), the aforementioned imaging techniques can be utilized to determine the extent of dissolution and/or the current status of the electrodes. Based on the imaging, remedial actions spanning from adjusting the current level and/or changing channels on a cochlear implant for example or otherwise adjusting the makeup of certain channels (e.g., using different electrodes to provide stimulation in a scenario where, all things being equal, the dissolved electrode(s) would otherwise be used) to explant in the electrode/device of which the electrode is apart and replacing it can be adopted and otherwise implemented.

Thus, the imaging techniques detailed herein can be utilized as a risk management technique. It is noted that the various embodiments detailed herein that utilize other regimes to evaluate the wear status and/or wear rate of the electrode can also be utilized as a risk management technique.

In an exemplary additional action of method 1700, there is the action of adjusting a parameter of the implant of which the electrode is apart. In an exemplary embodiment where the electrode is part of a cochlear implant, this additional action includes the action of adjusting a parameter of the cochlear implant, such as, for example, an electrical parameter (e.g., current application level). Additional exemplary embodiments of the adjustments that can be made based on the various determinations detailed herein or otherwise the analysis detailed herein will be described in greater detail below.

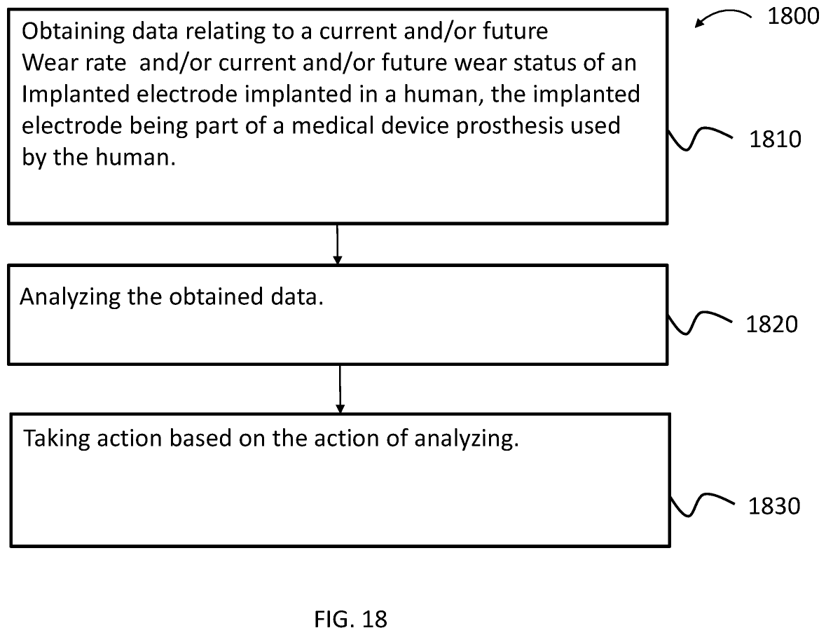

FIG. 18 presents an exemplary flowchart for an exemplary method, method 1800, which includes method action 1810, which includes obtaining data relating to a current and/or future passive dissolution and/or active dissolution rate and/or current and/or future passive dissolution and/or active dissolution status of an implanted electrode implanted in a human, the implanted electrode being part of a medical device prosthesis used by the human. In at least some exemplary embodiments, the action of obtaining can be executed utilizing any one or more of the methodologies detailed herein. Further, any methodology that can result in data that relates to the current and/or future passive dissolution and/or active dissolution rate and/or current and/or future passive dissolution and/or active dissolution status of an implanted electrode implanted in a human can be utilized in at least some exemplary embodiments, providing that such is utilitarian value in the art enable such.

Method 1800 further includes method action 1820, which includes analyzing the obtained data. As with all of the actions of analyzing detailed herein unless otherwise noted, the action of analyzing can be executed automatically and/or manually. Artificial intelligence can be utilized or otherwise a trained neural network or an expert system can be utilized. The action of analyzing the obtained data can include determining the wear rate and/or wear status of the electrode(s). The action of analyzing the obtained data can also include determining a believed causation of the passive dissolution and/or active dissolution of the electrode.

Method 1800 further includes method action 1830, which includes taking action based on the action of analyzing. In an exemplary embodiment, method action 1830 can correspond to identifying an adjustment of an operational parameter of the medical device prosthesis to, change, such as to slow a future rate of passive dissolution and/or active dissolution. In an exemplary embodiment, this can entail reducing a current applied by the electrode. Again, as promised, some additional ways to slow the future passive dissolution rate and/or active dissolution rate will be described.

Note also that the action of analyzing the obtained data can also include determining that there is no active dissolution and/or passive dissolution/the analysis can be to determine that there is no active dissolution and/or passive dissolution, and/or that any passive dissolution and/or active dissolution is de minimis, at least with respect to the ultimate timelines associated with need of the electrode (if the electrode wear rate that is determined would result in the electrode wearing out decades after the expected life expectancy of the user, such wear would be de minimis). In an exemplary embodiment, if indeed there is no passive dissolution and/or active dissolution or otherwise the rate is de minimis, method action 1830 can result in action can be taken that increases a wear rate but also increase the efficacy of the prostheses. This too would be a result of implementing an identified adjustment of an operational parameter of the medical device prostheses to change a future rate of passive dissolution and/or active dissolution. By way of example only and not by way of limitation, an increase in current amplitude and/or an increase in focusing of a multipolar stimulation can be used in at least some exemplary embodiments. While this may result in an increased wear rate, this can also result in superior performance results, such as the ability to evoke a hearing percept that more accurately represents normal hearing (how a person with normal hearing hears).

Method action 1830 can also include the action of prescribing a substance to be taken by the human (e.g., ingested, injected, inhaled, etc.) to slow the future rate of passive dissolution and/or active dissolution and/or proscribing a substance to be taken by the human that has an effect on the future rate of passive dissolution and/or active dissolution. Such substances could change the chemistry of body fluids, such as a chemistry of the perilymph, that has an effect on the passive dissolution and/or active dissolution rate.

In some embodiments, the recipient can be directed or otherwise adjusted to change treatments for certain conditions. Counseling can be provided regarding the use of drugs found to have a deleterious effect on the rate of passive dissolution and/or active dissolution of an electrode. Conversely, a recipient can be instructed or otherwise encouraged to utilize certain therapeutic substances, such as drugs, over-the-counter or prescriptive, etc., that can have a positive effect on the biochemistry of the recipient so as to reduce, which includes halting, the dissolution of the electrodes.

In yet another exemplary embodiment, method action 1830 can include instructing the human who is in receipt of the medical device prostheses to use the medical the device prostheses in a different manner. This could include, in the embodiment where the medical device is a cochlear implant, limiting the use of the cochlear implant relative to that which would otherwise be the case, such as, for example, utilizing the implant only when engaging in conversations. By limiting the amount of use of the implant, the longevity of the electrodes can be extended in at least some exemplary embodiments. Some additional ways to utilize the implant differently to change the passive dissolution and/or active dissolution rate will be described below.

It is also noted that the actions of method action 1830 can include the affirmative actions resulting from the identification and/or proscribing and/or proscribing and/or instructing. For example, instead of identifying an adjustment and/or including identifying an adjustment, the adjustment is also made.

It is also noted that in an exemplary embodiment, method action 1830 can include taking no action. In many exemplary scenarios, method actions 1810 and 1820 will be executed where the analysis of the obtained data results in a finding that the electrodes are not dissolving and/or eroding or otherwise that the rate of passive dissolution and/or active dissolution is de minimis. As will be detailed below, method actions 1810 and 1820 can be executed repeatedly and executed a number of times far greater than method action 1830 is executed.

In an exemplary embodiment where method action 1830 entails identifying an adjustment of an operational parameter of the medical device prostheses to slow a future rate of passive dissolution and/or active dissolution, where the medical device is a cochlear implant, the adjustment is a reduction in a degree of focusing for at least one channel of the cochlear implant which channel uses the electrode.

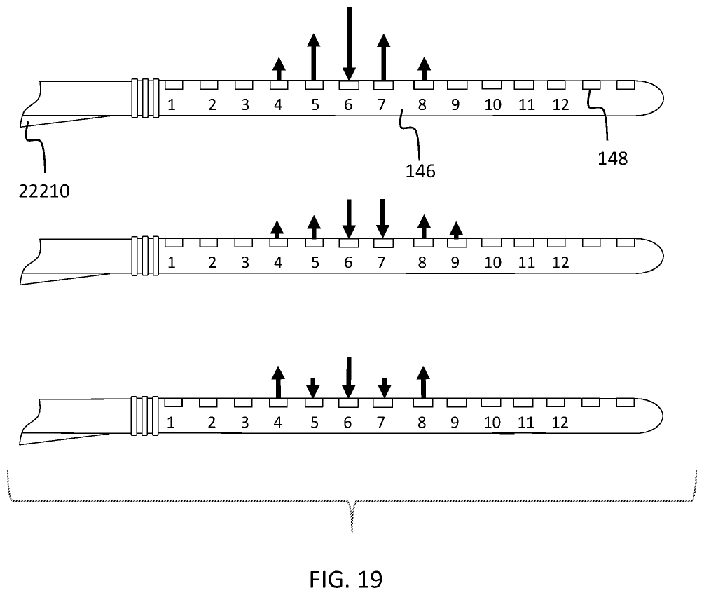

In an exemplary embodiment, say, where the results of the analysis of method action 1820 results in a determination rate that the dissolution rate on an electrode is too high (e.g., the current rate will result in the electrode dissolving before the end of the recipient's life), the stimulation is spread out in a focused multipolar mode or other modes to more than one electrode. FIG. 19 presents a schematic symbolically representing how current could be redistributed in a focused multipolar mode. As can be seen, there is an electrode array with a plurality of electrodes, of which 12 have been enumerated for the purposes of this discussion. It is to be noted that this is simply exemplary and that the actions associated with one electrode can be applicable to the other electrodes. It is also to be understood that other embodiments can handle a given electrode differently. In any event, it is noted that the size and directions of the arrows indicate the magnitude and direction of current, respectively. The top electrode array depicts current flow in a normal or otherwise an optimized setting (optimized for that particular recipient vis-à-vis the hearing percept evoked in the person—as distinguished from, for example, battery life or electrode longevity for example). Upon executing method action 1820, it is determined that the electrode 6 is experiencing a wear rate which is higher than that which is deemed acceptable. The focusing of the multipolar currents is adjusted to that seen in the middle electrode array. This has the effect of reducing the wear rate on electrode 6.

The bottom electrode array also depicts optimized focused multipolar currents for the optimization of a hearing percept. Again, where a determination is made that electrode 6 is dissolving or otherwise eroding at an unacceptably high rate, the focusing of the currents can be adjusted to the arrangement also seen in the middle or two another arrangement for that matter.

It is briefly noted that the diagram in FIG. 19 illustrates some exemplary embodiments of how charge can be rearranged in focused multipolar stimulation mode. Some embodiments utilize similar methods for other stimulation modes, such as by way of example only and not by way of limitation, monopolar mode (where instead of a single intracochlear electrode, two or more are used so as to distribute the charge) and/or bipolar mode (two intracochlear anodes, two intracochlear cathodes).

Accordingly, it can be seen that in at least some exemplary embodiments, there is the action of redistributing current in a focused multipolar mode. In some exemplary scenarios of this, there can be some loss of focusing, but this may be small or otherwise may result in little or no loss of performance. The benefit may outweigh disadvantage.

Some exemplary embodiments include performing a cost-benefit analysis, or more accurately, a benefit-disadvantage analysis, where it is deemed that the change in focusing or otherwise the adjustment would result in a decrease in the hearing performance of the cochlear implant, another type of stimulation change may instead be adopted or otherwise experimented with. If indeed the stimulation pattern cannot be changed to reduce the rate of passive dissolution and/or active dissolution in a utilitarian manner while still maintaining satisfactory results with respect to evoking a hearing percept, the adjustment may be abandoned and it may be accepted that at some point, that electrode will simply wear out.

In any event, at least some exemplary embodiments include reducing the maximum charge delivered to any single electrode, and thus reducing a dissolution rate exponentially with the decrease in delivered charge. By spreading the “center” charge of a stimulated electrode to neighboring electrodes, the overall dissolution rate can be decreased, in some embodiments, by significant amount.

In at least some exemplary embodiments where the action taken is the reduction of a degree of focusing for one or more or all channels (a cochlear implant can have 10, 11, 12, 13, 14, 15, 16, 17, 18, 19, 20, 21, 22, 23, 24, 25, 30, 35, 40, 45, 50, 60, 70, 80, 90, or 100 or more channels, or any value or range of values therebetween in one channel increments, and any one or more of these numbers can be adjusted according to the teachings detailed herein), the reduction reduces the stimulation amplitude from one or more of the electrodes associated with that channel (electrodes can be associated with one specific channel, or channels can make up a plurality of channels). The reduction in focusing, in some embodiments, is tuned to the rate of passive dissolution and/or active dissolution, and otherwise adjusted based on the additional data obtained from the recipient and/or other recipients that have a statistical correlation to the recipient or otherwise scenarios related to the recipient. In an exemplary embodiment, a stimulation rate of one or more of the electrodes can be reduced. In an exemplary embodiment, a degree of focusing and/or a stimulation rate is reduced by and/or equal to 5, 10, 15, 20, 25, 30, 35, 40, 45, 50, 55, 60, 65, 70, 75, 80, 85, 90, or 95% or more, or any value or range of values therebetween in 1% increments.

It is briefly noted that while most of the teachings detailed herein are directed towards taking action to reduce the active dissolution and/or passive dissolution rate, again, consistent with the teachings detailed above, in some instances, actions are taken that actually increase the wear rate of the electrode. This is done to improve the efficacy of the prosthesis, but limited in a manner that takes into account that the increased wear that results is still within the range that will enable a sufficient lifetime of use of the electrode. Accordingly, any disclosure herein of an action taken to increase the longevity or otherwise reduce the wear rate of an electrode corresponds to a disclosure of an alternate embodiment where the action is taken that actually decreases the longevity or otherwise increase the wear rate of the electrode.

In at least some exemplary embodiments, the actions taken to reduce the wear rate of the electrode can be any action that reduces the charge per phase, at least in a manner that has a significant results on the longevity of the electrode relative to that which would be the case in the absence of such reduction. In an exemplary embodiment, the reduction of charge per phase can be a reduction that is at least and/or equal to 5, 10, 15, 20, 25, 30, 35, 40, 45, 50, 55, 60, 65, 70, 75, 80, 85, 90, or 95% or more, or any value or range of values therebetween in 1% increments from the baseline.

In an exemplary embodiment, one or more channels can be disabled completely. In an exemplary embodiment, a recipient can actively enable the channel when needed, but otherwise that channel could be not used. In an exemplary embodiment, sound can be shifted to another channel, etc. This is the case with any one or more of the changes to the prostheses detailed herein. The prostheses can be configured to enter into an “optimum” mode or a “most desirable”/“most preferred” mode when the recipient once such or otherwise believes that it is necessary, but otherwise, the prostheses operates in the reduced functionality mode.

While the embodiments just described focus on adjustments to the operation of the hearing prosthesis, embodiments can include more pedestrian actions that prolong the longevity of electrode array, as noted above, such as for example, reducing the proportion of time and/or the number of listening environments in which focused stimulation is delivered. (e.g., focusing may not be used during quiet or in noise, or may not be used at all).

In an exemplary embodiment, by taking one or more of the actions associated with method action 1830 the other actions detailed herein, so as to change the passive dissolution rate and/or increase the longevity of the electrode, the dissolution rate is decreased by at least and/or equal to 10, 15, 20, 25, 30, 35, 40, 45, 50, 55, 60, 65, 70, 75, 80, 85, 90, or 95% or more, or any value or range of values therebetween in 1% increments. In an exemplary embodiment, the rate that constitutes the baseline is the average rate (mean and/or median) for the preceding month and/or quarter and/or year and/or 2 or 3 or 4 or 5 years or more or any value or range of values therebetween in one month increments, as measured from the change/adjustment, or otherwise the action associated with method action 1830. In an exemplary embodiment, the rate that constitutes the decreased rate is the average rate for the following month and/or quarter and/or year and/or 2 or 3 or 4 or 5 years or more or any value or range of values therebetween in one month increments, as measured from the change/adjustment, or otherwise the action associated with method action 1830.

In an exemplary embodiment, the estimated longevity and/or the actual longevity of the electrode, as a result of method action 1830 otherwise the actions detailed herein, is increased by at least and/or equal to 10, 20, 30, 40, 50, 75, 100, 150, 200, 250, 300, 350, 400, 450, 500, 600, 700, 800, 900, or 1000 percent or more, or any value or range of values therebetween in 1% increments. In an exemplary embodiment, the longevity that constitutes the baseline is the believed remaining longevity for that electrode at the time of the change/adjustment, or otherwise the action associated with method action 1830.

In an exemplary embodiment, such as where the medical device is a cochlear implant, the adjustment is a reduction in the stimulation rate of stimulation applied by the electrode. In an exemplary embodiment, the stimulation rate is decreased by at least and/or equal to 5, 10, 15, 20, 25, 30, 35, 40, 45, 50, 55, 60, 65, or 70% or more, or any value or range of values therebetween in 1% increments. In an exemplary embodiment, the rate that constitutes the baseline is the average rate (mean and/or median) for the preceding month and/or quarter and/or year and/or 2 or 3 or 4 or 5 years or more or any value or range of values therebetween in one month increments, as measured from the change/adjustment, or otherwise the action associated with method action 1830.

As noted above, in an exemplary embodiment, method action 1830 includes instructing the human to use the medical device prosthesis in a different manner. In an exemplary embodiment, such as where the medical device is a cochlear implant, the use of the cochlear implant in a different manner is the reduction of a proportion of time and/or a number of listening environments in which focused stimulation is delivered by the cochlear implant. In an exemplary embodiment, the instruction is to simply reduce the amount of time that the implant is utilized per day and/or per week, etc. In an exemplary embodiment, the instruction is to maintain a volume as low as possible or otherwise adjusted volume to a level lower than that which has historically been utilized by the recipient. In an exemplary embodiment, the instruction is to utilize stimulation modes that adopt a monopolar and/or a bipolar stimulation regime whenever possible or otherwise more than that which has historically been the case. In an exemplary embodiment, the instruction is to utilize as a default the stimulation regimes of lower complexity and/or less focus, and use the stimulation regimes of higher complexity and/or greater focus only on special occasions or otherwise as needed.

Of course, some exemplary methods include actually implementing the recommended changes.

It is briefly noted that at least method actions 1810 and 1820 can be executed a number of times during the lifetime of the implant. Method action 1830 can also be executed a number of times during the lifetime of the implant, however, it is expected that the number of times that method action 1830 will be executed will be lower than the number of times method actions 1810 and 1820 will be executed, owing to the fact that sometimes, the action of analyzing the obtained data will result in a determination that is at least inconclusive, if not indicative of the fact that the electrode is not dissolving or otherwise eroding, or otherwise dissolving and/or eroding at rates that are not deleterious or otherwise resulting in a need to implement method action 1830. Moreover, method action 1820 may not be executed as often as method action 1810, because the data may be collected at a number of different instances prior to the action of analyzing. For example, the data can be collected once a month over the course of the year, and thus method action 1810 would be executed 12 times in a year, and then at the end of the year, method action 1820 is executed only once.

Accordingly, in an exemplary embodiment, in a given temporal period, the method actions 1810, 1820 and/or 1830 can be executed more than and/or equal to 1, 2, 3, 4, 5, 6, 7, 8, 9, 10, 11, 12, 13, 14, 15, 20, 25, 30, 35, 40, 45, 50, 55, 60, 65, 70, 80, 90, 100, 125, 150, 175, 200, 300, 400, 500, 600, 700, 800, 900, 1000, 1250, 1500, 1750, 2000, 3000, 4000 or 5000 or 6000 or 7000 or 8000 or 9000 or 10000 times or more or any value or range of values therebetween in 1 increment. The timing between these events can be equally/evenly spaced. As noted above, the execution of method 1800 can be executed repeatedly where method action 1830 entails taking no action. Hence the value of some of the above noted numbers.

The aforementioned temporal time periods can be less than or equal to 1, 2, 3, 4, 5, 6, 7, 8, 9, 10, 11, 12, 13, 14, 15, 20, 25, 30, 35, 40, 45, 50, 55, 60, 65, 70, 80, 90, 100, 125, 150, 175, 200, 300, 400, 500, 600, 700, 800, 900, or 1000 months, or any value or range of values therebetween in 1 day increments.

In some exemplary embodiments, method action 1830 (and/or method action 2020 detailed below) where method action 1830 includes taking some form of action (as opposed to taking no action), may be executed at least and/or equal to 1, 2, 3, 4, 5, 6, 7, 8, 9, 10, 11, 12, 13, 14, 15, 20, 25, 30, 35, 40, 45, 50, 55, 60, 65, 70, 80, 90, or 100 times or more or any value or range of values therebetween in 1 increment. In an exemplary embodiment, the temporal periods between executing one or more or all of the method actions of method 1800 can be 1, 2, 3, 4, 5, 6, 7, 8, 9, 10, 11, 12, 13, 14, 15, 20, 25, 30, 35, 40, 45, 50, 55, 60, 65, 70, 80, 90, 100, 125, 150, 175, 200, 300, 400, or 500 or more days, and/or weeks, or any value or range of values therebetween in 1 day increments (this is also the case with respect to the actions of method 2000 detailed below). Thus, it can be seen that, for example, iterative techniques may be utilized to find out how to adjust the operation of the implant to obtain utility with respect to reducing the rate of passive dissolution and/or active dissolution or otherwise maintaining the longevity of the electrode for the needed temporal period. Further, in some embodiments, the adjustments will be changed to alter the efficacy of the prostheses. For example, after one or more adjustments, it may be the case that the dissolution rate has been reduced to a satisfactory level and/or even to a level much below that which would otherwise be satisfactory. However, the efficacy of the performance of the prostheses for its underlying purpose (e.g., the vocation of a hearing percept in the case of a cochlear implant) may not be that which would be desired or otherwise could be much better, and thus additional adjustments might be made that are directed towards efficacy of the underlying purpose of the prostheses.

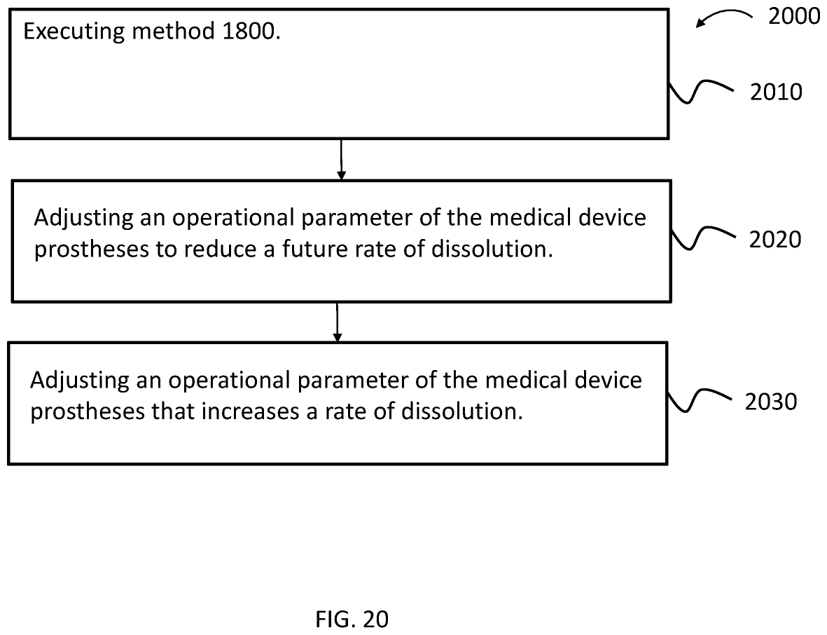

Accordingly, in an exemplary embodiment, there is an exemplary method, such as method 2000, as represented by the flowchart of FIG. 20, that includes method action 2010, which includes executing method 1800. Method 2000 further includes method action 2020, which includes adjusting an operational parameter of the medical device prostheses to reduce a future rate of passive dissolution and/or active dissolution. Method 2000 further includes method action 2030, which includes adjusting an operational parameter of the medical device prostheses that increases a future rate of passive dissolution and/or active dissolution. This is done in coordination to increase or otherwise improve the efficacy of the underlying medical device prostheses relative to that which was the case with respect to method action 2020. Here, in this embodiment, the active dissolution and/or passive dissolution rates have been stabilized or otherwise reduced to acceptable values, and in method action 2030, the goal is to improve the underlying efficacy of the prostheses, even though that might increase the passive dissolution and/or active dissolution rates. In an exemplary embodiment, the results of method action 2030, however any times that action is executed, results in an increase in the dissolution rate by rates that can correspond to those detailed above with respect to the reduction, however, here, the rates are increased (the text is not repeated in the interest of textual economy). And to be clear, consistent with at least some exemplary scenarios of the utilization of the teachings detailed herein, it could be that the active dissolution and/or passive dissolution rate is reduced to an acceptable level, but the prostheses does not have sufficient efficacy, and thus the prostheses must be adjusted to improve the efficacy, but that results in passive dissolution rates and/or active dissolution rates that increase to potentially that which was the case prior to the actions that resulted in the decrease, and, in some embodiments, actually even further increase the rate of dissolution beyond that which was the case without adjustment at all. The underlying goal is the efficacy of the prosthesis with respect to its original purpose.

It is also noted that in at least some exemplary embodiments, the results of the actions detailed herein may result in failure, even under the most inspirational definition thereof: where your absolute best just is not good enough. In this regard, it simply may be the case that there is just nothing that one can do to achieve sufficient longevity of the electrodes with respect to the desired usage times thereof. Even if the device was never used again, in some scenarios for example, the body chemistry might just eat up the electrodes. It is the proverbial the way it is. Thus, in a further variation of method 1800, the method further includes the action of subsequently obtaining second data relating to a subsequent current and/or subsequent future passive dissolution and/or active dissolution rate and/or subsequent current and/or subsequent future passive dissolution and/or active dissolution status of the implanted electrode implanted in the human, analyzing the subsequently obtained data and recommending at least one of a revision surgery to replace the electrode or implantation of a second medical device to supplement the medical device. In an exemplary embodiment, this “subsequent” set of actions can be executed after any of the number of times that the other actions of method 1800 are executed, as detailed above. In an exemplary embodiment, method 1800 could have been executed five or 10 or 20 or 30 times in totality, or some of those portions could have been executed as many times a just mentioned or many more times. In any event, at some point there is a determination that the electrode(s) cannot be saved/that the desired life of the electrode is not going to be met by the actual or estimated life of the electrode. Accordingly, depending on how much of a problem this is (problem with respect to the efficacy of the underlying medical device for its intended purpose) the component and/or device of which the electrode is a part could be explanted, and/or in some scenarios, a new component with new electrodes can be implanted. Still further, in scenarios where, for example, it is possible to simply add a new medical device or a new component, instead of explanting the component with the electrode, another component/device is implanted. With regard to the embodiment just described, this could entail adding a second side cochlear implant or a second side hearing prostheses to supplement the deficiencies and/or the soon-to-be deficiencies of the first implant. For example, if the trajectory of the failure mode vis-à-vis wear of the electrode(s) will result in, for example, the recipients of a cochlear implant not being able to have a hearing percept of frequencies within/between 700 Hz and 2000 Hz, by way of example only and not by way of limitation, the additional implant on the other side of the ear could supplement this deficiency of the currently implanted hearing prostheses. And to be clear, with respect to the embodiments detailed above, another possible course of action would be to simply utilize different electrodes and/or different channels to evoke a hearing percept at different frequencies than that upon which the sound is based. For example, a 1000 Hz ambient sound would evoke a hearing percept of a 2200 Hz sound for example, if the electrodes and/or channels for evoking such a precept are still functioning. Granted, the frequency of the sound may be different but the underlying content can still be understood. And that is the underlying goal of communication, where the dramatic effect lost in such a transposition would be a secondary and a relatively minor concern. Conversely, utilizing the second (additional) cochlear implant, the sound would be conveyed using that new implant and the old implant would simply not function for those frequencies or otherwise would not evoke a hearing percept for those frequencies (e.g., the electrodes can be deactivated and/or the channels for those frequencies can be deactivated). In an exemplary embodiment, there is no deactivation per se; the current applied to or otherwise applied to the dissolved/eroded electrodes simply does not evoke a hearing percept. Still, there can be utilitarian value with respect to taking more of an affirmative action with regard to recognizing that certain channels will no longer provide stimulation.

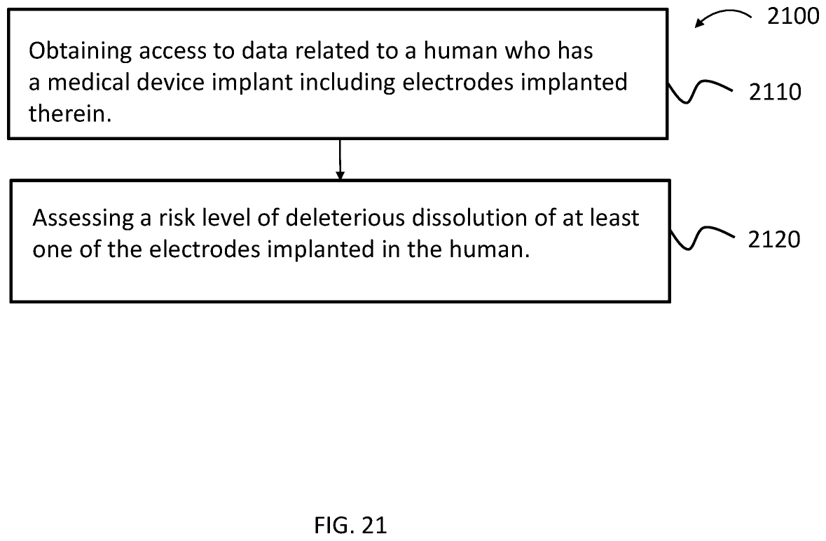

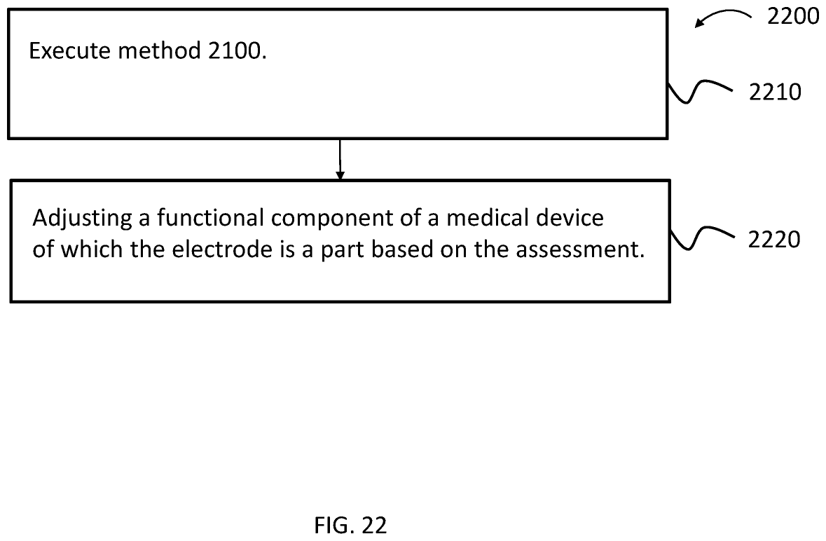

FIG. 21 depicts an exemplary algorithm for another method, method 2100, according to an exemplary embodiment. Method 2100 includes method action 2110, which includes the action of obtaining access to data related to a human who has a medical device implant including electrodes implanted therein. In an exemplary embodiment, this could entail obtaining the logged data logged by the medical device. This could be uploaded from the implantable component and/or the external component, during this action 2110, or could be data that has previously been conveyed, such as over the Internet to a remote database or the like. This could also include the hearing prosthesis obtaining the underlying data relating to the state of the electrodes for example (the transimpedance data for example). This can also entail obtaining medical records of the person who has the recipient. Any device and/or system that will enable method action 2110 and/or any action that can be taken to obtain method action 2110 can be utilized in at least some exemplary embodiments.

Method 2100 further includes method action 2120, which includes the action of accessing a risk level of deleterious passive dissolution and/or active dissolution of at least one of the electrodes implanted in the human. In an exemplary embodiment, the assessment of a risk level is executed based on at least one of:
statistical data applicable to the human;
physiological data pertaining to the human;
historical data pertaining to the human;
usage data pertaining to the electrode;
electrical phenomenon pertaining to the electrode;
spatial data pertaining to the electrode; or
design and/or performance data relating to the electrode.

In an exemplary embodiment, the assessments of risk level and/or the other determinations disclosed herein can be based on, for example, age and/or lifestyle of the recipient, such as, for example, the occupation of the recipient, a typical noise environment of the recipient and/or a noise environment of the recipient that is statistically aberrant relative to other average users of the cochlear implant. Biological features can include the composition of the perilymph, and thus comparisons can be made to a specific recipient's perilymph and the perilymph data on statistically significant individuals.

The assessment of risk level and/or other determinations disclosed herein can be based on electrode type and/or electro position in the cochlea (e.g., depth of an electrode, proximity to structures within the cochlea, such as by way of example only and not by way of limitation, the spiral ligament and/or modiolus, etc.) All of this data can be compared to statistically significant data and thus based on the comparison, assessed the risk level and/or otherwise make the other determinations disclosed herein.

Consistent with the teachings detailed herein, the risk level is used in at least some exemplary embodiments to select stimulation parameters (e.g., stimulation rate, pulse width, degree of focusing, etc.) that appropriately balance passive dissolution/active dissolution risk with other considerations such as hearing performance, sound quality, and/or otherwise preference of a particular hearing prostheses recipient. Accordingly, embodiments include an implantable prostheses, such as a hearing prostheses, that has been adjusted or otherwise include settings that take into account longevity of the electrodes thereof, and otherwise reduce the likelihood of premature wear out in a statistically significant manner beyond that which would otherwise be the case without this adjustment and/or without these settings. In at least some exemplary embodiments, the settings/adjustments reduce the efficacy of the hearing prostheses, sometimes in a noticeable and/or statistically significant manner, and otherwise result in a hearing prostheses that is not as efficacious as it could be with the settings, but which settings prolong the longevity of at least one of the electrodes of the electrode array.

Consistent with the teachings above, embodiments take into account recipient specific factors that change over the lifetime of the recipient. By way of example only and not by way of limitation, such factors that can change can result from the onset of unrelated disease and/or changing needs for medication. Some classes of drugs, presently in existence and/or envisioned or otherwise which may exist in the future, can impact the inner ear biochemistry, and dissolution. The teachings detailed herein can take that into account or otherwise compensate for such to provide a holistic treatment of a human being.

All of the above said, there are some instances where preference can dominate. For example, recipients may desire an increased volume. The fact that the sound is not as precise or otherwise as focused as it could be secondary. Accordingly, the focusing could be reduced to accommodate this increase in amplitude of the current so as to maintain the life expectancy of the cochlear implant electrode(s). And in this regard, it is to be understood that many embodiments herein are directed towards preemptory actions, where the electrodes at issue may not have experienced any significant wear and/or even noticeable/detectable wear.

It is noted that the action 2120 requires that a risk level of the deleterious state of the electrode he assessed. This is different than simply determining that there is some passive dissolution and/or active dissolution. This is different than an inconclusive “maybe” or “the data is inconclusive.” An actual risk level must be assessed. This is also different than simply determining that no passive dissolution and/or active dissolution has taken place. The fact that something has not happened does not mean that it will not happen.

In an exemplary embodiment, the result of method action 2120 is an assessment that there is a heightened risk level of deleterious passive dissolution and/or active dissolution. In an exemplary embodiment, this determination is relative to a statistically significant set of data for a population that includes in a relevant manner the human in which the implant is implanted.

It is noted that the nature of a risk level is forward-looking. That is, method action 2120 does not simply assess the current state of the electrode. The fact that the electrode has experienced passive dissolution and/or active dissolution does not in and of itself result in the assessment of a risk level. That said, such can be utilized in the overall action of assessing a risk level.

It is also noted that method action 2120 can be executed after one or more of the adjustments and/or actions regarding method 1800 have been executed and/or executed after one or more of those actions have been contemplated. That is, method action 2120 can entail contemplating adjustments to a hearing prosthesis for example, and taking into account those adjustments in the overall assessment of a risk level.

In an exemplary embodiment, the assessed risk level can be a three level scale (e.g., high, medium, low). In an exemplary embodiment, the assessed risk level can be a percentile ranking (e.g., 90% or 70 to 85.5% chance that the electrode will dissolve within ten years and/or within the lifetime of the recipient, a relative ranking—the data is indicative of statistical data were a certain action has occurred within five or 10 or 15 years, etc.). In an exemplary embodiment, the accessed risk level can be on a scale of 1 to 5 or 1 to 10, or a likelihood, etc. The assessed risk level can be that action should be taken or no action should be taken.

As noted above, the assessment of risk level can be based on statistical data applicable to the human. By way of example only and not by way of limitation, use characteristics of the implant (e.g., how much time in a given hearing environment, volume, usage amounts, types of usage (focused stimulation) and how much, etc.) can be compared to a statistical database for electrodes of the design and/or of a similar design. The database could include placement of the electrodes and/or locations of the electrodes, etc. Any statistical data applicable to the human that can aid in forecasting the risk level can be utilized in at least some exemplary embodiments, providing that such has utilitarian value in the overall assessment as opposed to having no correlation to the ultimate result.

Also as noted above, physiological data pertaining to the human can be obtained. This can include blood chemistry for example. Historical data pertaining to the human can be also utilized, such as, for example, how the recipient has used a hearing prosthesis in the past, the medical history of the recipient, how long the recipient has had the implant, the age of the recipient, the current health status of the recipient (if the recipient may not live for more than 20 years, a dissolution rate that would result in a deleterious event 22 years hence may not be a risk).

It is noted that some of the bases listed above are not mutually exclusive from others. For example, the assessment of risk level can be based on usage data pertaining to the electrode. This could be included in data regarding how the human utilizes the implant. This is not to say that it is included, it is just to say that it could be included. For example, if the data pertaining to how the human utilizes the implant indicates that the human utilizes the implant to listen to native French language speakers in a statistically significant manner for example, it can be deduced that the electrode relating to the higher frequencies are used more often than that which was otherwise the case. Still, this would require an affirmative determination of the relationship to that specific electrode.

In general, uses data pertaining to the electrode can include the type of polar stimulation (monopolar, bipolar, tripolar, etc.), the current level and/or the phase and/or the pulse width etc.

In an exemplary embodiment, with respect to electrical phenomenon pertaining to the electrode, this can correspond to the voltage readings and/or current readings and/or impedance readings, etc., by way of example only and not by way of limitation.

With respect to spatial data pertaining to the electrode, this can correspond to the location of the electrodes within the body or and/or the proximity of the electrodes to tissue structures within the body, etc. With respect to design data relating to the electrode, the design features of a given electrode can be obtained, either from the original design drawling/specifications for example, or deduced from inspection (e.g., reverse engineering, such as, for example, from high-resolution images of the actual implanted electrodes, and/or knowledge relating to other produced medical devices corresponding to the implanted medical device, which other produce medical devices are available for inspection, etc.). With respect to performance data, this can entail utilizing historical data relating to a given electrode design. By way of example only and not by way of limitation, if an implant corresponding to design X is known to have features related to electro-dissolution, that information can be utilized to assess the risk level of the implanted electrodes. This could be utilitarian if the implant corresponds to the design X, or if the electrodes implanted in the human are of the same electrodes were at least similar electrodes as in design X. Moreover, long-term data development can be utilized in conjunction with the implanted devices. By way of example, a design/production run for a given implant can be utilized in practice and implanted in humans starting on date Z. At the same time or proximate that time, or at a time before or after that has utilitarian value, a similar product and/or design can be tested or otherwise utilized as a control. For example, a cochlear implant electrode array can be placed into a model cochlea and the electrodes thereof could be stimulated in a manner that can to how those electrodes would be utilized over two or three or four or five or 10 or 20 years or more. The electrodes of the control/model can be analyzed, and the data resulting from the analysis could be utilized as performance data relating to the actual implanted electrodes. The point is, in view of the long-term nature of the issues associated with electro-dissolution, a long term control experiment can be utilized during temporal periods that at least overlap the temporal periods associated with the actual electrodes being implanted in actual humans, and the data from the experiment can be utilized in the methods detailed herein.

In an exemplary embodiment, the action of assessing the risk level is based on at least two or more of the following:
a size and/or shape of the cochlea;
a lifestyle of the human recipient of the implant;
composition of perilymph;
diseases and/or co-morbidities and/or associated treatments;
cochlear implant design and/or cochlear implant surgical factors;
electrode type; or
electrode position in the cochlea.

Consistent with the details above, the action of assessing the risk level can be executed by a neural network or other expert system.

Corollary with the teachings detailed herein, in an exemplary embodiment, there is an exemplary method, method 2200, which includes method action 2210, which includes executing method 2100. Method action 2200 further includes method action 2220, which includes adjusting a functional component of a medical device of which the electrode is a part based on the assessment of method action 2120.

In an exemplary embodiment, the action of adjusting a functional component of a medical device of which the electrode is a part based on the assessment is such that the action of adjusting is executed, as measured from a date of implantation of the electrode, within and/or equal to 5, 10, 15, 20, 25, 30, 35, 40, 45, 50, 55, 60, 65, 70, 75, 80, 85, or 90%, or any value or range of values therebetween in 1% increments of a reliability engineering based average (mean, median and/or mode) design life expectancy of the implantable portion of the medical device. Reliability engineering based design life expectancies are not specifically usage based. That is, one takes the design and determine the life expectancy based on normal usage. If abnormal usage occurs, the reliability might be recalculated, but that is usage based reliability. Thus, for example, for a model ABC prosthesis, the reliability engineering based average design life expectancy could be 75 years (can be the case for an implantable component of a hearing prostheses such as a cochlear implant). The reliability engineering based average design life expectancy could be 50 years for pacemaker (e.g., because people would require pacemakers later in life). This is all as contrasted to, for example, that which may be the case if the implantable component is utilized in an abnormal manner. By way of example only and not by way of limitation, if a user of the cochlear implant always has the volume to the maximum, the current amplitude of electrical signals applied to the electrodes will be higher on average than that which would otherwise be the case. This could cause the electrodes to wear faster than the average user of a cochlear implant. Reliability engineering data can exist for such usage, but that is not design usage, as the implant is not designed for use at a maximum volume for 50 years, even though it could be utilized at a maximum volume for a relatively long time, and there could be a possibility that the device would not wear out within that time period.

With respect to the above noted percentages of the life expectancy, if, for example, a cochlear implant has a life expectancy of 50 years, or more accurately, if the implantable portion of the cochlear implant, has a life expectancy of 50 years, or even more accurately, if the electrode array of the cochlear implant has a life expectancy of 50 years (the receiver stimulator of the cochlear implant could potentially be replaced without disturbing the electrode array or otherwise replacing the electrode array—moving the electrode array could cause problems with respect to the cochlea's accommodation of an existing array—a new receiver stimulator could be implanted and attached to the old electrode array in general, and the leads coming from the electrode array in particular, in at least some exemplary scenarios), and the adjustment is made within 10% of the reliability engineering based average design life expectancy of the implantable portion and/or the electrode array for that matter, the adjustment would be made within five years. The adjustment could be made within two years or three years.

In an exemplary embodiment, the action of assessing the risk level can be executed at least 1, 2, 3, 4, 5, 6, 7, 8, 9, 10, 11, 12, 13, 14, 15, 20, 25, 30, 35, 40, 45, 50, 60, 70, 80, 90, 100, 125, 150, 175, 200, 300, 400, 500, 600, 700, 800, 900, 1000, 1500, 2000, 3000, 4000, 5000, or any value or range of values therebetween in 1 increments, within any of the temporal periods detailed herein, and/or within the lifetime of the implanted electrode and/or within the reliability engineering based average design life expectancy of the medical device and/or implant and/or electrode. Some of these numbers are high relatively speaking, and thus it can be understood the utilitarian value of utilizing artificial intelligence and/or automated techniques. In this regard, the assessment of risk level can be executed utilizing a trained neural network, and such a network could assess the risk once a day or more over a century or more potentially.

Consistent with the teachings detailed above, the action of adjusting a functional component can include adjusting a functional component of a cochlear implant of which the electrode is a part based on the assessment, wherein the adjustment reduces a performance qualify of the cochlear implant relative to that which was the case prior to the adjustment. In this regard, by way of example, the focusing could be broadened, thus reducing the overall sound quality of the hearing percept evoked by a cochlear implant, but increasing the longevity of a given electrode at issue.

It is briefly noted that in at least some exemplary methods, the methods explicitly exclude explanting the electrode(s). In an exemplary embodiment, at least some of the analysis and/or determinations detailed herein are based on data that has nothing to do with an explanted electrode(s), at least not the electrode(s) at issue of the analysis. Accordingly, embodiments can enable the analyses and/or determinations to be made without having to explant the electrode(s) and/or otherwise physically access the electrode(s).

In an exemplary embodiment, the electrodes at issue are utilized/have been utilized to apply bipolar and/or tripolar and/or multipolar stimulation for at least and/or equal to 1, 2, 3, 4, 5, 6, 7, 8, 9, 10, 11, 12, 13, 14, 15, 20, 25, 30, 35, 40, 45, or 50 years or more, or any value or range of values therebetween in one month increments, at the time that at least one or more of the actions detailed herein are executed. In an exemplary embodiment, there are cochlear implant electrode arrays that have implanted electrode(s) that have been used to apply any one or more of the simulations detailed herein for at least and/or equal to 1, 2, 3, 4, 5, 6, 7, 8, 9, 10, 11, 12, 13, 14, 15, 20, 25, 30, 35, 40, 45, or 50 years or more, or any value or range of values, to evoke a hearing percepts at least once a week or at least once a month during that time. Any one or more of the method actions can be executed during any one or more of those time periods. It is further noted that in at least some exemplary embodiments, the cochlear implant electrode array have at least 50, 60, 70, 80, 81, 82, 83, 84, 85, 86, 87, 88, 89, 90, 91, 92, 93, 94, 95, 96, 97, 98, 99, or 100%, or any value or range of values therebetween in 1% increments of the original electrodes remaining operational to evoke a hearing percepts. Accordingly, in at least some exemplary embodiments, the teachings detailed herein can be utilized to increase the longevity of an electrode beyond that which would otherwise have been the case in the absence of the teachings detailed herein.

Some embodiments specifically do not rely on estimates of population averages of wearout rates, etc. That is, some exemplary embodiments do not rely on statistical analysis, or at least statistical data does not drive the ultimate conclusions and/or assessments and/or determinations. By way of example only and not by way of limitation, data obtained by utilizing the composite electrodes detailed above can control the determination. By way of example, even if the statistical data indicates that there is not a problem or otherwise that the wear rate is within acceptable levels, other data as disclosed herein can drive the determination. Accordingly, embodiments include discounting or otherwise taking action that is diametrically opposed to what statistical data would indicate based on a sample population, etc. Of course, as noted above, embodiments include a combination of data where a holistic approach is utilized. In some embodiments, some data can be weighted over other data. For example, the statistical data could have a weighting of one, and the electrical phenomenon associated with the composite electrodes can have a weighting of three for example.

Exemplary embodiments include management of life changes. For example, in at least some exemplary embodiments, a stronger/higher magnitude current will be utilitarian as the recipient ages, where, the increased current will result in a faster dissolution rate of the electrode. In an exemplary embodiment, an embodiment takes into account these changes, and proactively estimate the ultimate life expectancy of the electrode array. For example, all things being equal, statistical data based on the future hearing prostheses device needs of a recipient can be utilized to forecast future required for utilitarian settings of a given hearing prostheses. The teachings detailed herein can be utilized to provide an estimate of the remaining useful life of the electrode (taking into account, for example, a safety factor where an electrode must have at least 20 or 15% or so of its remaining mass or volume or thickness to be reliably useful). It could be that it is foreseen that the electrode will have to be explanted or otherwise changed out or otherwise will not last for its needed useful life. It could be that a remedial action now as opposed to in the future would be more utilitarian. By way of example, a surgery when a person is younger is typically “safer” than that which would be the case when the recipient gets older. In another exemplary embodiment, it could be that the electrode is “saved” 41 it is later needed. That is, for example, the performance of the implant could be reduced now so that higher performance could be utilized later, when it is needed. For example, the magnitude of current applied to a given electrode in the short run can be purposely reduced beyond that which would otherwise be the case and could provide a minor inconvenience, balance out by the fact that in later years, a higher current will be needed. The idea being that the electrodes are managed so as to achieve their desired otherwise needed life. Thus, embodiments disclosed herein include a management regime that can span years and/or decades, or any of the temporal periods detailed herein, where the actions taken are not only based on presents usages, but also can be based on future uses of the prostheses.

Some embodiments include testing or otherwise evaluating each of the electrodes of an implant. Conversely, some embodiments include testing only some of the electrodes out of the total number of electrodes. By way of example only and not by way of limitation, some embodiments will result in data being available that indicates which electrodes are utilized more than others in or otherwise which electrodes would statistically likely have a greater degree of wear relative to others. By way of example only and not by of limitation, the electrodes associated with speech frequencies would likely be experiencing greater wear than the electrodes for frequencies higher than the average speech frequencies. That said, in embodiments where, for example, the speech frequencies are spread out over a number of electrodes, and the higher frequencies are concentrated on a single electrode, the reverse can be the case. A noise or sound environment of the recipient can be evaluated. Data relating to the stimulation arrangements or otherwise the parameters of the implant can be evaluated. For example, if it is known that a given electrode experiences highly focused and/or high magnitude currents of short pulse durations, some embodiments include identifying that electrode as a leading candidate for early passive dissolution and/or active dissolution. Accordingly, at least some embodiments can include concentrating the evaluations on that electrode. Indeed, some embodiments can be such that it is not possible to evaluate or otherwise analyzed or otherwise monitor some electrodes. Still, in some embodiments, all of the electrodes can be monitored or otherwise analyzed.

At least some exemplary embodiments according to the teachings detailed herein utilize advanced techniques to analyze the data to forecast or otherwise ascertain a dissolution rate and/or a status of the solution, which are able to be trained or otherwise are trained to detect higher order, and/or non-linear statistical properties of the data inputted into the system, which data can correspond to any one or more of the examples detailed herein and/or any other that can have utilitarian value to ultimately forecast or otherwise ascertain or estimate a rate of passive dissolution/active dissolution and/or a status of the passive dissolution/active dissolution. An exemplary data processing technique is the so called deep neural network (DNN). At least some exemplary embodiments utilize a DNN (or any other advanced learning data processing technique) to process data, which processed data is utilized to evaluate the electrodes according to the teachings herein. At least some exemplary embodiments entail training data processing algorithms to process data to implement at least some of the exemplary methods herein. That is, some exemplary methods utilize learning algorithms or regimes or systems such as DNNs or any other system that can have utilitarian value where that would otherwise enable the teachings detailed herein to analyze the data relating to the electrodes.

Embodiments include utilizing a so-called “neural network” that can be a specific type of machine learning system. Any disclosure herein of the species “neural network” constitutes a disclosure of the genus of a “machine learning system.” While embodiments herein focus on the species of a neural network, it is noted that other embodiments can utilize other species of machine learning systems. Accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Embodiments include method actions associated with processes to train DNNs so as to enable those DNNs to be utilized to execute at least some of the method actions detailed herein.

It is noted that in at least some exemplary embodiments, the DNN or the product from machine learning, etc., is utilized to achieve a given ability to evaluate/process the data detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product from machine learning, etc., that when used, results in that functionality, unless otherwise noted or unless the art does not enable such.

Exemplary embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network.

All the above said, in some embodiments, standard processors that are programmed in the traditional manner/that are not machine learning based and/or chips that are formatted in a traditional manner and include logic circuitry that are configured to execute at least some of the exemplary method actions detailed herein are utilized. Computers that are programmed or otherwise configured to accept the data and/or retrieve the data and/or process the data or otherwise evaluate the data can be utilized to execute at least some of the method actions detailed herein. Note also that any reference to a method action herein that is implemented utilizing artificial intelligence and/or a neural network and/or machine learning corresponds to an alternate embodiment where the reference is to a functionality of a device. By way of example only and not by way of limitation, in an exemplary embodiment, if there is a disclosure herein of a medical device that is configured to evaluate the data relating to the electrodes and determine a wear rate and/or wear status of the electrodes, such corresponds to a disclosure of utilizing a product of machine learning to analyze that data, where the product of the machine learning can be a computer chip for example, which computer chip is part of the medical device.

It is noted that any method action disclosed herein corresponds to a disclosure of a non-transitory computer readable medium that has program there on a code for executing such method action providing that the art enables such. Still further, any method action disclosed herein where the art enables such corresponds to a disclosure of a code from a machine learning algorithm and/or a code of a machine learning algorithm for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. That is, as will be detailed below, a neural network can be “fed” significant amounts (e.g., statistically significant amounts) of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code—code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A medical device, comprising:
an implantable portion of the medical device, the implantable portion including at least one electrode, wherein
the implantable portion is configured to, while implanted in a human, obtain data indicative of wear of the at least one electrode.

2. The medical device of claim 1, wherein:
the implantable portion is configured to communicate the obtained data and/or data based on the obtained data transcutaneously to a device located outside the human.

3. The medical device of claim 1, wherein:
the medical device is configured to analyze the obtained data and determine a wear status of the at least one electrode and communicate an indication of the wear status.

4. The medical device of claim 1, wherein:
the medical device is configured to enable an adjustment of an operation of the cochlear implant to reduce a future wear rate of the at least one electrode and/or one or more other electrodes of the implantable component.

5. (canceled)

6. The medical device of claim 1, wherein:
the medical device is configured to analyze the obtained data and determine a wear status of the at least one electrode and automatically adjust an operation of the cochlear implant to compensate for the wear of the at least one electrode.

7. The medical device of claim 1, wherein:
the medical device is a cochlear implant.

8. The medical device of claim 1, wherein:
the medical device is configured to analyze the obtained data and determine a wear status of the at least one electrode and automatically recommend an action to reduce a future passive dissolution and/or active dissolution rate of the at least one electrode and/or one or more other electrodes of the implantable component and/or recommend an adjustment operation of the cochlear implant to compensate for the wear of the at least one electrode.

9-10. (canceled)

11. An implantable electrode array, comprising:
a plurality of electrodes; and
a carrier carrying the plurality of electrodes, wherein
the implantable electrode array is configured to enable in vivo analysis of a wear status of at least one electrode of the plurality of electrodes.

12. The implantable electrode array of claim 11, wherein:
the implantable electrode array is configured to provide an abrupt change in an electrical phenomenon upon one or more electrodes of the plurality of electrodes reaching a wear status.

13. (canceled)

14. The implantable electrode array of claim 11, wherein:
the at least one electrode has a first layer of material and a second layer of material different from the first layer of material, the second layer of material being below the first layer of material relative to an ambient environment of the electrode array, the different layers enabling the in vivo analysis.

15. The implantable electrode array of claim 11, wherein:
the at least one electrode has a first layer and a second layer having a different structure than the first layer, the second layer of material being below the first layer of material relative to an ambient environment of the electrode array, the different structure enabling the in vivo analysis.

16. The implantable electrode array of claim 11, wherein:
the at least one electrode has a first layer and a second layer;
the first layer has a porosity different from the second layer;
the second layer is below the first layer relative to an ambient environment of the electrode array; and
the different porosity enabling the in vivo analysis.

17. The implantable electrode array of claim 11, wherein:
the at least one electrode has a first layer and a second layer;
the first layer is made of different material and/or has a porosity different from the second layer;
the second layer is below the first layer relative to an ambient environment of the electrode array; and
the different material and/or different porosity enabling the in vivo analysis.

18. (canceled)

19. The implantable electrode array of claim 11, wherein:
the at least one electrode is configured to trigger a measurable change in a cyclic voltammetry spectrum of the electrode upon a wear amount of the at least one electrode, the measurable change enabling the in vivo analysis.

20-37. (canceled)

38. A non-transitory computer-readable media having recorded thereon, a computer program for executing at least a portion of a hearing-prosthesis fitting method, the computer program including:
code for obtaining data related to a human who has a medical device implant including electrodes implanted therein; and
code for assessing a risk level based on the obtained data, of deleterious passive dissolution and/or active dissolution of an implanted electrode implanted in a human based on at least one of:
statistical data applicable to the human;
physiological data pertaining to the human;
historical data pertaining to the human;
usage data pertaining to the electrode;
electrical phenomenon pertaining to the electrode;
spatial data pertaining to the electrode; or
design and/or performance data relating to the electrode.

39. The media of claim 38, wherein:
the electrode is implanted in a cochlear of the human, and is part of a cochlear implant.

40. The media of claim 39, wherein:
the action of assessing a risk level of deleterious passive dissolution and/or active dissolution of an implanted electrode implanted in the human is based on at least two or more of:
a size and/or shape of the cochlea;
a lifestyle of the human;
composition of perilymph;
diseases and/or co-morbidities and/or associated treatments;
cochlear implant design and/or cochlear implant surgical factors;
electrode type; or
electrode position in the cochlea.

41. The media of claim 38, wherein:
the media is used by and/or in a neural network.

42. The media of claim 38, further comprising:
code for identifying an adjustment of a functional component of a medical device of which the electrode is a part based on the assessment.

43. The media of claim 38, further comprising:
code for identifying an adjustment of a functional component of a cochlear implant of which the electrode is a part based on the assessment, wherein the adjustment reduces a performance qualify of the cochlear implant relative to that which was the case prior to the adjustment.

44-47. (canceled)
